(19) United States
(12) Patent Application Publication
(10) Pub. No.: US 2024/0158350 A1
(43) Pub. Date: May 16, 2024

(54) ESTER ANALOGS OF PSILOCIN, PROCESSES FOR THE PREPARATION THEREOF, AND METHODS OF USE

(71) Applicant: Invyxis, Inc., New York, NY (US)

(72) Inventors: Jeffrey O'MEARA, Chertsey (CA); Harpreet KAUR, Brampton (CA); Ahmed Magdy ALI, Milton (CA); Peter Martin DOVE, Burlington (CA)

(21) Appl. No.: 18/487,841

(22) Filed: Oct. 16, 2023

Related U.S. Application Data
(60) Provisional application No. 63/416,250, filed on Oct. 14, 2022.

(51) Int. Cl. C07D 209/20; C07D 405/12; C07D 409/12

(52) U.S. Cl. CPC C07D 209/20; C07D 405/12; C07D 409/12

(57) ABSTRACT

The disclosure relates to compounds, compositions, methods and uses relating to activation of one or more serotonin receptors (i.e., 5-HT2 receptors). Specifically, the disclosure provides ester derivatives of psilocin and related tryptamines, and methods for treating neurological diseases or disorders, including neurodegenerative diseases, pain, and/or psychiatric disorders that comprise the compounds or compositions thereof. Also provided are methods for preparing the compounds described herein.

### CROSS-REFERENCE TO RELATED APPLICATIONS

This application claims the benefit of U.S. Provisional Application No. 63/416,250, filed Oct. 14, 2022, which is hereby incorporated by reference in its entirety for all purposes.

### BACKGROUND

Despite the increased interest and attention given to areas such as mental health and opioid abuse, there remains a need for safe and effective pharmacotherapies, especially for pain, severe depression, treatment resistant depression, and psychological distress related with life-threatening diseases (among others).

Substituted tryptamine alkaloids such as psilocin are known to exert pharmacological effects through the binding and activation of serotonin receptors (i.e., 5-HT2_{A}, 5-HT2_{B}, and 5-HT2_{C}). Serotonergic psychedelic compounds have been demonstrated to be useful in the treatment and management of a number of mental health conditions, for example as antidepressants, anti-anxiety/anxiolytics, and anti-addiction agents.

Nevertheless, there remains a need to generate new analogs of tryptamine alkaloids (i.e., psilocins) that are able to exhibit serotonergic effects and/or provide favorable properties relating to one or more of selectivity, bioavailability, pharmacokinetic (PK), and/or pharmacodynamic (PD) properties relative to psilocin. The disclosure provides such compounds.

### SUMMARY OF THE DISCLOSURE

In an aspect the disclosure provides psilocin analogs that are effective agonists for one or more serotonin receptors.

In an aspect the disclosure provides psilocin analogs that are effective agonists for one or more serotonin receptors.

In an aspect, the disclosure provides a compound, or derivative thereof, of Formula (I):

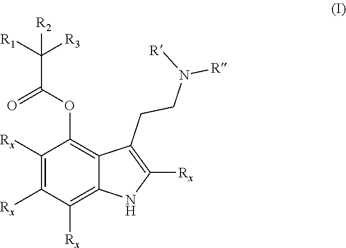

wherein R₁ is absent or comprises hydrogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R₂ and R₃ independently comprise hydrogen, deuterium, halogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl; or
R₂ and R₃ together with the carbon atom to which they are attached form a C₃-C₂₀ cycloalkyl, aryl, or heteroaryl, any of which may be optionally substituted with one or more halogen, C₁-C₆ alkyl, C₂-C₆ alkenyl, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl; and
each Rₓ is independently H, —OH, methyl, methoxy, C₁-C₃ alkyl, C₁-C₆ haloalkyl, deuterated C₁-C₃ alkyl, C₁-C₃ alkoxy, —C(═O)OH, —C(═O)NR₂R₃, or halogen;
and wherein the compound is not

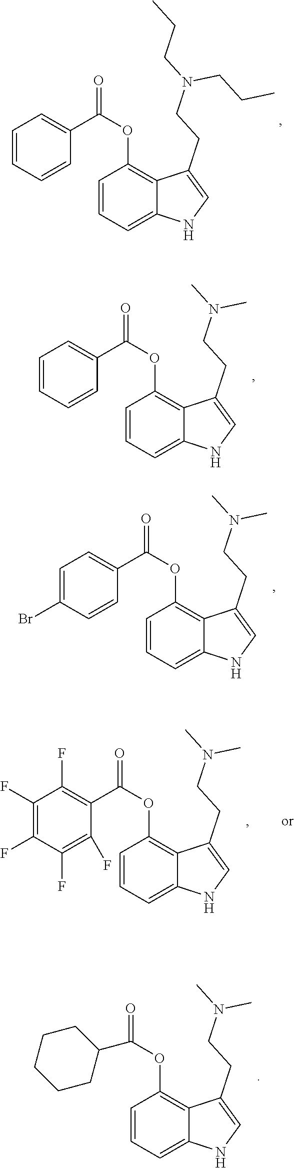

In embodiments relating to compounds of Formula (I), R′ and R″ independently comprise C₁-C₆ alkyl.

In embodiments relating to compounds of Formula (I), R₁, R₂, and R₃ do not comprise C₁-C₁₂ alkyl.

In embodiments relating to compounds of Formula (I), each Rₓ independently comprises H, —OH, methyl, methoxy, or halogen.

In embodiments relating to compounds of Formula (I), R₁ is absent and R₂ and R₃ with the carbon atom to which they are attached form a C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl.

In embodiments relating to compounds of Formula (I), (i) one of R₂ or R₃ comprises hydrogen, and the other of R₂ or R₃ comprises C₂-C₆ alkenyl, C₂-C₆ alkynyl, C₁-C₆ alkoxy, or C₃-C₁₂ cycloalkyl; or (ii) R₂ and R₃ with the carbon atom to which they are attached form a C₃-C₁₂ cycloalkyl that is optionally substituted.

In embodiments relating to compounds of Formula (I), the compounds comprise Formula (Ib):

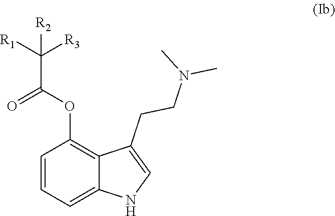

or pharmaceutically acceptable salt, ester, amide, and prodrug thereof, wherein
R₁ is absent and R₂ and R₃ with the carbon atom to which they are attached form a C₃-C₂₀ cycloalkyl, aryl, or heteroaryl, any of which may be optionally substituted with one or more halogen, C₁-C₆ alkyl, C₂-C₆ alkenyl, C₁-C₆ haloalkyl, C₃-C₁₂ cycloalkyl, C₃-C₁₂ heterocyclyl, aryl, or heteroaryl.

In embodiments relating to compounds of Formula (I), the compound is:
3-(2-(dimethylamino)ethyl)-1H-indol-4-yl furan-3-carboxylate;
3-(2-(dimethylamino)ethyl)-1H-indol-4-yl 2-fluorobenzoate;
3-(2-(dimethylamino)ethyl)-1H-indol-4-yl 3-fluorobenzoate;
3-(2-(dimethylamino)ethyl)-1H-indol-4-yl 4-fluorobenzoate; or
3-(2-(dimethylamino)ethyl)-1H-indol-4-yl thiophene-2-carboxylate.

In another aspect, the disclosure provides a compound, or derivative thereof, of Formula (II):

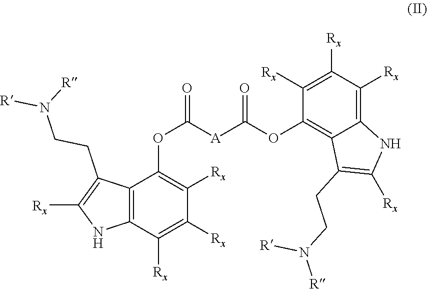

wherein A is C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl;
each Rₓ is independently H, —OH, methyl, methoxy, C₁-C₃ alkyl, C₁-C₆ haloalkyl, deuterated C₁-C₃ alkyl, C₁-C₃ alkoxy, —C(═O)OH, —C(═O)NR₂R₃, or halogen.

In embodiments relating to compounds of Formula II, R′ and R″ independently comprise C₁-C₆ alkyl.

In embodiments relating to compounds of Formula II, each Rₓ is independently H, —OH, methyl, methoxy, or halogen.

In embodiments relating to compounds of Formula II, A comprises C₁-C₆ alkyl, C₂-C₆ alkenyl, C₂-C₆ alkynyl, C₁-C₆ alkoxy, or C₃-C₁₂ cycloalkyl.

In embodiments relating to compounds of Formula II, the compound comprises a structure according to Formula (IIb):

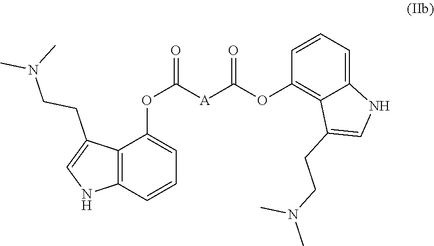

or pharmaceutically acceptable salt, ester, amide, and prodrug thereof, wherein

A comprises C₁-C₆ alkyl, C₂-C₆ alkenyl, C₂-C₆ alkynyl, or C₁-C₆ alkoxy, C₃-C₁₂ cycloalkyl, C₃-C₁₂ heterocyclyl, aryl, or heteroaryl.

In embodiments relating to compounds of Formula (II), the compound is: bis(3-(2-(dimethylamino)ethyl)-1H-indol-4-yl) isophthalate;
bis(3-(2-(dimethylamino)ethyl)-1H-indol-4-yl) 4,6-dimethylisophthalate;
bis(3-(2-(dimethylamino)ethyl)-1H-indol-4-yl) (1S,2S)-cyclopropane-1,2-dicarboxylate;
bis(3-(2-(dimethylamino)ethyl)-1H-indol-4-yl) 2,2-dimethylmalonate;
bis(3-(2-(dimethylamino)ethyl)-1H-indol-4-yl) succinate;
bis(3-(2-(dimethylamino)ethyl)-1H-indol-4-yl) glutarate; or
bis(3-(2-(dimethylamino)ethyl)-1H-indol-4-yl) adipate.

In embodiments of any of the compounds of the above described aspects and embodiments can comprise at least one deuterium substitution. In some further embodiments, or in some alternative embodiments, of any of the compounds of the above described aspects and embodiments can comprise at least one halogen substitution.

In another aspect the disclosure provides a pharmaceutical composition, comprising any of the compounds of the above described aspects and embodiments and a pharmaceutically acceptable carrier.

In another aspect the disclosure provides a method for treating one or more conditions that are responsive to serotonin receptor activation, comprising administering to a subject in need thereof an effective amount of a compound of any of the above described aspects and embodiments.

In another aspect the disclosure provides a method for treating a neurological disease, comprising administering to a subject in need thereof an effective amount of a compound of any of the above described aspects and embodiments. In further embodiments, the neurological disease comprises a neurodegenerative disease, stupor and coma, dementia, seizure, sleep disorder, trauma, infection, neoplasm, neuro-ophthalmological condition, movement disorder, demyelinating disease, spinal cord disorder, disorder of peripheral nerves, muscle and neuromuscular junctions, psychiatric disorder, or pain, or a disease associated with pain. In yet further embodiments, the psychiatric disorder comprises an anxiety disorder including acute stress disorder agoraphobia, generalized anxiety disorder, obsessive-compulsive disorder, panic disorder, posttraumatic stress disorder, separation anxiety disorder, social phobia, or specific phobia; a childhood disorder including attention-deficit/hyperactivity disorder, conduct disorder, or oppositional defiant disorder; an eating disorder including anorexia nervosa or bulimia nervosa; a mood disorder including depression, bipolar disorder, cyclothymic disorder, dysthymic disorder, or major depressive disorder; a personality disorder including antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, or schizotypal personality disorder; a psychotic disorder including brief psychotic disorder, delusional disorder, schizoaffective disorder, schizophreniform disorder, schizophrenia, or shared psychotic disorder; a substance-related disorder including alcohol dependence, amphetamine dependence, cannabis dependence, cocaine dependence, hallucinogen dependence, inhalant dependence, nicotine dependence, opioid dependence, phencyclidine dependence, or sedative dependence; an adjustment disorder, autism, delirium, dementia, multi-infarct dementia, a learning or memory disorder including amnesia or age-related memory loss; or Tourette's disorder. In some further embodiments, the neurological disease is pain, or is a disease associated with pain.

Additional aspects and embodiments in accordance with the disclosure will be apparent to one of skill in the art in light of the following description.

### BRIEF DESCRIPTION OF THE DRAWINGS

FIGS. 1A-B depict pharmacokinetic data of test compounds A (FIG. 1A) and B (FIG. 1B) (ng/mL v. hr.) in vivo.

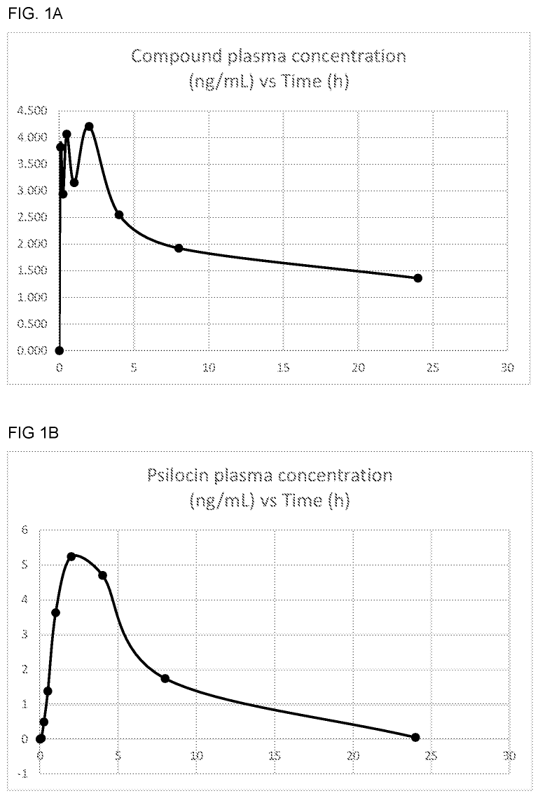

### DETAILED DESCRIPTION OF EMBODIMENTS

Before the disclosed methods and materials are described, it is to be understood that the aspects described herein are not limited to specific embodiments, and can vary. It is also to be understood that the terminology used herein is for the purpose of describing particular aspects only and, unless specifically defined herein, is not intended to be limiting.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The following terms and expressions used herein have the indicated meanings.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety.

An “alkyl” group refers to a fully saturated straight or branched chain hydrocarbon containing from 1 to 12 carbon atoms, which is attached to a molecule by a single bond. Alkyl groups can include C₁-C₁₂ alkyl, C₁-C₁₀ alkyl, C₁-C₆ alkyl, C₁-C₅ alkyl all of which are inclusive of C₄ alkyls, C₃ alkyls, C₂ alkyls and C₁ alkyl (methyl). Non-limiting examples of alkyl include methyl, ethyl, n-propyl, iso-propyl, n-butyl, sec-butyl, iso-butyl, tert-butyl, n-pentyl, isopentyl, neopentyl, t-amyl, n-hexyl, 3-methylhexyl, 2,2-dimethylpentyl, 2,3-dimethylpentyl, n-heptyl, n-octyl, n-nonyl, n-decyl, n-undecyl, and n-dodecyl. In accordance with some example embodiments an alkyl group can be optionally substituted.

“Alkylene” refers to a saturated, straight or branched bivalent alkyl group. An “alkylene chain” refers to a polymethylene group, i.e., —(CH₂)ₙ—, wherein n is a positive integer which, in certain embodiments, can be from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkenyl” refers to a straight or branched chain hydrocarbon containing from 2 to 12 carbons and containing at least one carbon-carbon double bond. Alkenyl groups can include C₂-C₁₂ alkenyl, C₂-C₁₀ alkenyl, C₂-C₆ alkenyl, C₂-C₅ alkenyl all of which are inclusive of C₄ alkenyls, C₃ alkenyls, and C₂ alkenyls. Non-limiting examples of alkenyl include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3- pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. In accordance with some example embodiments an alkenyl group can be optionally substituted.

The term “alkynyl” refers to a straight or branched chain hydrocarbon group containing from 2 to 12 carbon atoms and containing at least one carbon-carbon triple bond. Alkynyl groups can include C₂-C₁₂ alkynyl, C₂-C₁₀ alkynyl, C₂-C₆ alkynyl, C₂-C₅ alkynyl all of which are inclusive of C₄ alkynyl, C₃ alkynyl, and C₂ alkynyl. Non-limiting examples of alkynyl include, but are not limited, to acetylenyl (ethynyl), propynyl (i.e., 1-propynyl, 2-propynyl), butynyl, pentynyl, and the like. In accordance with some example embodiments an alkynyl group can be optionally substituted.

“Alkoxy” refers to a group of the formula —OR, where R is an alkyl, alkenyl, or alkynyl group, as defined herein, appended to the parent molecular moiety through the oxygen atom. Non-limiting examples of alkoxy groups include methoxy, ethoxy, propoxy, 2-propoxy, butoxy, tert-butoxy, pentyloxy, and hexyloxy. In accordance with some example embodiments an alkoxy group can be optionally substituted.

The term “aryl” refers to a stable monocyclic (i.e., phenyl), bicyclic, tricyclic or tetracyclic ring system containing 6 to 18 carbon atoms and at least one aromatic ring in the ring system. An aryl group can include fused and/or bridged ring systems. Non-limiting examples of aryl include aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In accordance with some example embodiments an aryl group can be optionally substituted.

The term “cycloalkyl” refers to a stable monocyclic, bicyclic, polycyclic, or spirocyclic fully saturated ring system typically comprising from 3 to 20 carbon atoms. Monocyclic ring systems are cyclic hydrocarbon groups that In embodiments contain from 3 to 10 carbon atoms. Non-limiting examples of monocyclic cycloalkyls include cyclopropyl, cyclobutyl, cyclopentyl, cyclopentenyl, cyclohexyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Non-limiting examples of bicyclic and polycyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane, adamantyl, norbornyl, decalinyl, 7,7-dimethyl-bicyclo[2.2.1]heptanyl, and the like. In accordance with some example embodiments a cycloalkyl group can be optionally substituted.

“Cycloalkenyl” refers to a stable non-aromatic monocyclic, bicyclic, or polycyclic hydrocarbon consisting solely of carbon and hydrogen atoms, having one or more carbon-carbon double bonds, which can include fused or bridged ring systems, having from 3 to 20 carbon atoms, preferably having from 3 to 10 carbon atoms. Monocyclic cycloalkenyls include, for example, cyclopentenyl, cyclohexenyl, cycloheptenyl, cycloctenyl, and the like. Unless otherwise stated specifically in the specification, a cycloalkenyl group can be optionally substituted.

The term “halo” or “halogen” refers to one or a combination of —Cl, —Br, —I, or —F.

The terms “haloalkyl,” “haloalkenyl,” “haloalkynyl,” and “haloalkoxy” refer to an alkyl, alkenyl, alkynyl, or alkoxy group, as defined above, which is substituted with one or more halogen atoms at any available position. In accordance with some example embodiments any of these “halo-” groups can be optionally substituted.

“Heteroaryl,” refers to a 5- to 20-membered ring system such as a monocyclic, bicyclic, tricyclic, or tetracyclic ring system that can be fused or bridged, and that contains at least one aromatic ring and that includes one to six heteroatoms selected from oxygen, nitrogen, and sulfur. Monocyclic heteroaryl groups can suitably be a 5- or 6-membered ring. A bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When a bicyclic heteroaryl contains a fused cycloalkyl, cycloalkenyl, or heterocyclyl ring, the bicyclic heteroaryl group can be connected to the parent molecular moiety through any carbon or nitrogen atom contained within the monocyclic heteroaryl portion of the bicyclic ring system. When a bicyclic heteroaryl is a monocyclic heteroaryl fused to a benzo ring, then the bicyclic heteroaryl group can be connected to the parent molecular moiety through any carbon atom or nitrogen atom within the bicyclic ring system. Non-limiting examples of heteroaryls include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). In accordance with some example embodiments a heteroaryl group can be optionally substituted.

The terms “heterocyclyl” and “heterocycle” refer to a 3- to 20- membered monocyclic, bicyclic, polycyclic, or spirocyclic ring system that may be saturated, unsaturated, or aromatic and that includes from 1 to 6 heteroatoms, N, O, or S. Monocyclic heterocycles include 3, 4, 5, 6, and 7 membered-rings containing at least 1 heteroatom independently selected from the group consisting of O, N, and S. The heterocycle is connected to the parent molecular moiety through any carbon atom or any nitrogen atom contained within the heterocycle. Non-limiting examples of monocyclic heterocycles include azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. Non-limiting examples of bicyclic heterocycles include 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl.

The term “oxo” as used herein means a ═O, group.

The term “thia” as used herein means a ═S group.

The term “saturated” as used herein means the referenced chemical structure does not contain any multiple carbon-carbon bonds. For example, a saturated cycloalkyl group as defined herein includes cyclohexyl, cyclopropyl, and the like.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

In view of the present disclosure, the methods and compositions described herein can be configured by the person of ordinary skill in the art to meet the desired need. In general, the disclosed materials and methods provide improvements in treatment of neuorological and neurodegenerative diseases, including pain and mental health/psychiatric disorders. The disclosed materials and methods also generally provide for improved agonists that are selective for particular 5-HT2 receptors.

### Compounds

### I. Ester Compounds

In one aspect, the disclosure provides compounds of Formula (I):

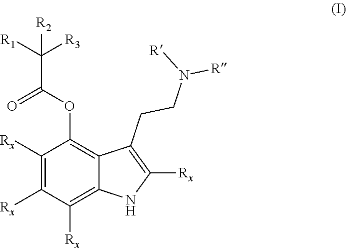

and/or derivatives thereof, or pharmaceutically acceptable salts, esters, amides, and prodrugs thereof, wherein
R₁ is absent or comprises hydrogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R₂ and R₃ independently comprise hydrogen, deuterium, halogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl; or
R₂ and R₃ together with the carbon atom to which they are attached form a C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl; and
each Rₓ is independently H, —OH, methyl, methoxy, C₁-C₃ alkyl, deuterated C₁-C₆ alkyl, C₁-C₃ alkoxy, —C(═O)OH, —C(═O)NR₂R₃, or halogen.

In embodiments, the present disclosure provides compounds of Formula (I), wherein R₁ is absent or comprises hydrogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R₂ and R₃ independently comprise hydrogen, deuterium, halogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl; or
R₂ and R₃ together with the carbon atom to which they are attached form a C₃-C₂₀ cycloalkyl, aryl, or heteroaryl, any of which may be optionally substituted with one or more halogen, C₁-C₆ alkyl, C₂-C₆ alkenyl, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl; and
each Rₓ is independently H, —OH, methyl, methoxy, C₁-C₃ alkyl, C₁-C₆ haloalkyl, deuterated C₁-C₆ alkyl, C₁-C₃ alkoxy, —C(═O)OH, —C(═O)NR₂R₃, or halogen; and wherein the compound is not

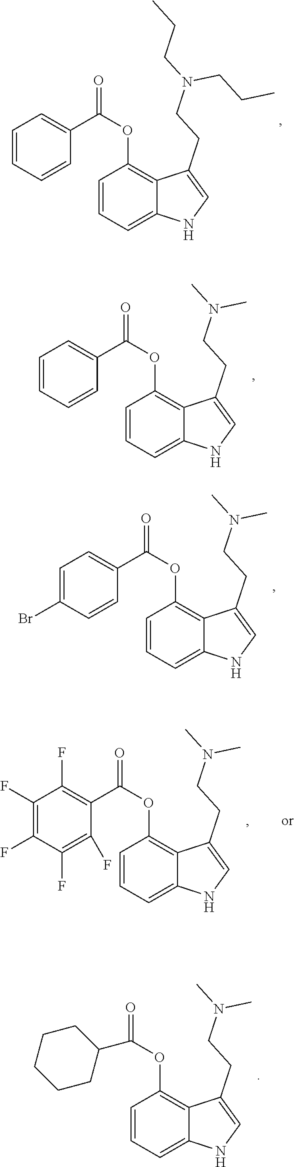

For purposes of clarity, the numbering convention for the indole ring that may be referred to in various aspects and embodiments of the compounds is presented below with respect to the structure of Formula (I):

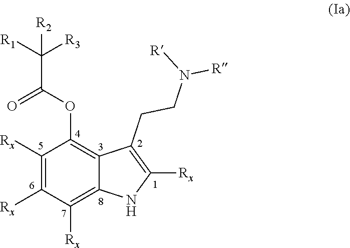

Some embodiments of the disclosure provide compounds of Formula (I) as otherwise described herein where R₁ is C₁-C₆ alkyl, C₂-C₆ alkenyl, C₂-C₆ alkynyl, C₁-C₆ alkoxy, or C₃-C₁₂ cycloalkyl. In some further embodiments, R₁ is C₁-C₆ alkyl or C₁-C₆ alkoxy. In some alternative embodiments of the disclosure, the compounds of Formula (I) are as otherwise described herein where R₁ is C₃-C₁₂ heterocyclyl, aryl, or heteroaryl.

Some embodiments of the disclosure provide compounds of Formula (I) as otherwise described herein, but wherein one, two, or all of R_{1,} R_{2,} and R₃ do not comprise C₁-C₁₂ alkyl.

Some embodiments of the disclosure provide compounds of Formula (I) as otherwise described herein where R₂ and R₃ independently comprise hydrogen, C₁-C₆ alkyl, C₂-C₆ alkenyl, C₂-C₆ alkynyl, C₁-C₆ alkoxy, or C₃-C₁₂ cycloalkyl. In some further embodiments, the compounds of Formula (I) comprise a structure wherein one or R₂ or R₃ comprise hydrogen, and the other of R₂ and R₃ comprise C₁-C₆ alkyl, C₂-C₆ alkenyl, C₂-C₆ alkynyl, C₁-C₆ alkoxy, or C₃-C₁₂ cycloalkyl. In yet further embodiments, the compounds of Formula (I) comprise a structure wherein one of R₂ or R₃ comprise hydrogen, and the other of R₂ and R₃ comprise C₁-C₆ alkyl, C₁-C₆ alkoxy, or C₃-C₁₂ cycloalkyl. In some alternative embodiments, the compounds of Formula (I) comprise a structure wherein one of R₂ or R₃ comprise hydrogen, and the other of R₂ and R₃ comprise C₁-C₆ haloalkyl, C₃-C₁₂ heterocyclyl, aryl, heteroaryl. In yet further embodiments, the disclosure provides compounds of Formula (I) as otherwise described herein, where R₂ and R₃ with the carbon atom to which they are attached form a C₃-C₁₂ cycloalkyl or C₃-C₁₂ heterocyclyl that is optionally substituted.

In any of the aspects and embodiments described above, some compounds of the disclosure include R′ and R″ as independently selected from H and C₁-C₆ alkyl. In embodiments R′ and R″ are independently selected from C₁-C₆ alkyl. In further embodiments, R′ and R″ are independently selected from C₁-C₄ alkyl. In embodiments, R′ and R″ comprise the same C₁-C₄ alkyl group. In embodiments, R′ and R″ comprise different C₁-C₄ alkyl groups. In any of the above embodiments, R′ and R″ can comprise unsubstituted C₁-C₄ alkyl groups (e.g., methyl, ethyl, propyl, isopropyl, n-butyl, isobutyl, or t-butyl groups). In embodiments, compounds of Formula (I) comprise a structure as otherwise defined herein, wherein at least one of R′ and R″ comprise a methyl group. In further embodiments, compounds of Formula (I) comprise a structure wherein each R′ and R″ comprise a methyl group.

In any of the aspects and embodiments described above, some compounds of the disclosure include each Rₓ as independently selected from H, —OH, methyl, methoxy, C₁-C₃ alkyl, C₁-C₃ alkoxy, or halogen. In embodiments, at least one Rₓ comprises a halogen. In some further embodiments, the halogen comprises Cl or F. In embodiments Rₓ at ring position 2 comprises methyl. In embodiments, compounds of Formula (I) comprise one or more Rₓ group that is hydrogen. In embodiments, compounds of Formula (I) comprise a structure as otherwise defined herein, wherein wherein each Rₓ group is hydrogen.

In some non-limiting example embodiments, the compounds of the disclosure comprise a structure according to Formula (Ib):

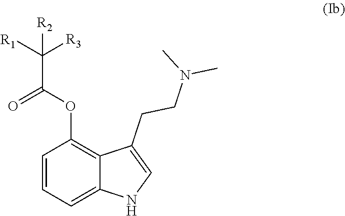

or pharmaceutically acceptable salts, esters, amides, and prodrugs thereof, wherein
R₁ is absent and R₂ and R₃ with the carbon atom to which they are attached form a C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl.

In certain non-limiting example embodiments, the compounds are selected from any one or more of the following 3-(2-(dimethylamino)ethyl)-1H-indol-4-yl 4-bromobenzoate; 3-(2-(dimethylamino)ethyl)-1H-indol-4-yl furan-3-carboxylate; 3-(2-(dimethylamino)ethyl)-1H-indol-4-yl 2-fluorobenzoate; 3-(2-(dimethylamino)ethyl)-1H-indol-4-yl 3-fluorobenzoate; 3-(2-(dimethylamino)ethyl)-1H-indol-4-yl 4-fluorobenzoate; or 3-(2-(dimethylamino)ethyl)-1H-indol-4-yl thiophene-2-carboxylate:

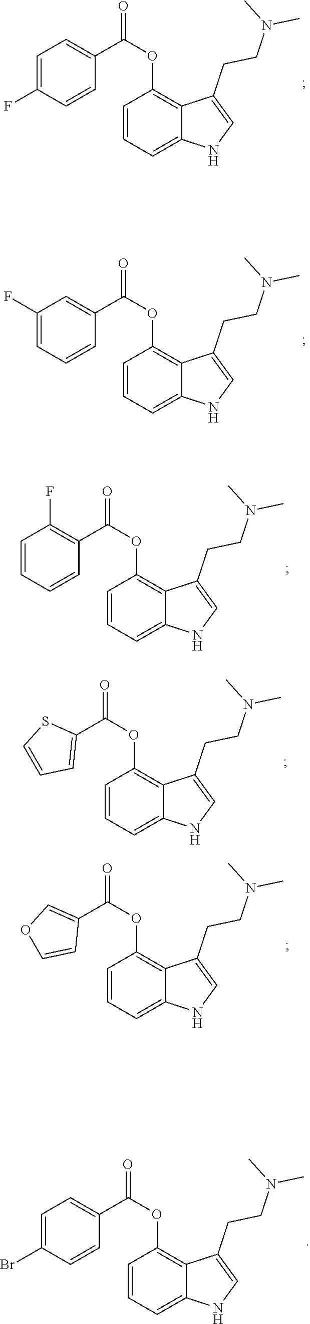

In embodiments, the compound is 3-(2-(dimethylamino)ethyl)-1H-indol-4-yl furan-3-carboxylate. In embodiments, the compound is 3-(2-(dimethylamino)ethyl)-1H-indol-4-yl 2-fluorobenzoate. In embodiments, the compound is 3-(2-(dimethylamino)ethyl)-1H-indol-4-yl 3-fluorobenzoate. In embodiments, the compound is 3-(2-(dimethylamino)ethyl)-1H-indol-4-yl 4-fluorobenzoate. In embodiments, the compound is 3-(2-(dimethylamino)ethyl)-1H-indol-4-yl thiophene-2-carboxylate.

### II. Psilocin Bridged and Dimer Compounds

In one aspect, the disclosure provides compounds of Formula (II):

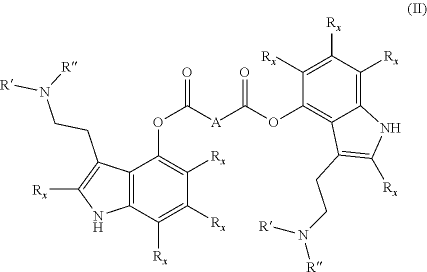

and/or derivatives thereof, or pharmaceutically acceptable salts, esters, amides, and prodrugs thereof, wherein
A is C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl;
each Rₓ is independently H, —OH, methyl, methoxy, C₁-C₃ alkyl, C₁-C₆ haloalkyl, deuterated C₁-C₃ alkyl, C₁-C₃ alkoxy, —C(═O)OH, —C(═O)NR₂R₃, or halogen.

For purposes of clarity, the numbering convention for the indole ring that may be referred to in various aspects and embodiments of the compounds is presented below with respect to the structure of Formula (IIa):

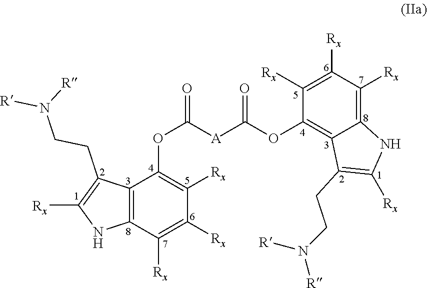

Some embodiments of the disclosure provide compounds of Formula (II) as otherwise described herein where A is C₁-C₆ alkyl, C₂-C₆ alkenyl, C₂-C₆ alkynyl, C₁-C₆ alkoxy, or C₃-C₁₂ cycloalkyl. In some further embodiments, A is C₁-C₆ alkyl or C₁-C₆ alkoxy. In some alternative embodiments of the disclosure, the compounds of Formula (II) are as otherwise described herein where A is C₃-C₁₂ heterocyclyl, aryl, or heteroaryl.

In any of the aspects and embodiments described above, some compounds of the disclosure include R′ and R″ as independently selected from H and C₁-C₆ alkyl. In embodiments R′ and R″ are independently selected from C₁-C₆ alkyl. In further embodiments, R′ and R″ are independently selected from C₁-C₄ alkyl. In embodiments, R′ and R″ comprise the same C₁-C₄ alkyl group. In embodiments, R′ and R″ comprise different C₁-C₄ alkyl groups. In any of the above embodiments, R′ and R″ can comprise unsubstituted C₁-C₄ alkyl groups (e.g., methyl, ethyl, propyl, isopropyl, n-butyl, isobutyl, or t-butyl groups). In embodiments, compounds of Formula (II) comprise a structure as otherwise defined herein, wherein at least one of R′ and R″ comprise a methyl group. In further embodiments, compounds of Formula (II) comprise a structure wherein each R′ and R″ comprise a methyl group.

In any of the aspects and embodiments described above, some compounds of the disclosure include each Rₓ as independently selected from H, —OH, methyl, methoxy, C₁-C₃ alkyl, C₁-C₃ alkoxy, or halogen. In embodiments, at least one Rₓ comprises a halogen. In some further embodiments, the halogen comprises Cl or F. In embodiments Rₓ at ring position 2 comprises methyl. In embodiments, compounds of Formula (II) comprise one or more Rₓ group that is hydrogen. In embodiments, any compounds of Formula (II) comprise a structure as otherwise defined herein, wherein each Rₓ group is hydrogen.

In embodiments, the compounds of the disclosure comprise a structure according to Formula (IIb):

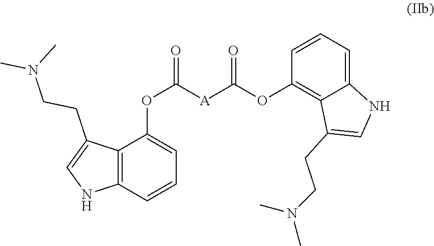

or pharmaceutically acceptable salts, esters, amides, and prodrugs thereof, wherein
A is C₁-C₆ alkyl, C₂-C₆ alkenyl, C₂-C₆ alkynyl, or C₁-C₆ alkoxy, C₃-C₁₂ cycloalkyl, C₃-C₁₂ heterocyclyl, aryl, or heteroaryl.

In embodiments, the compounds are any one or more of the following:

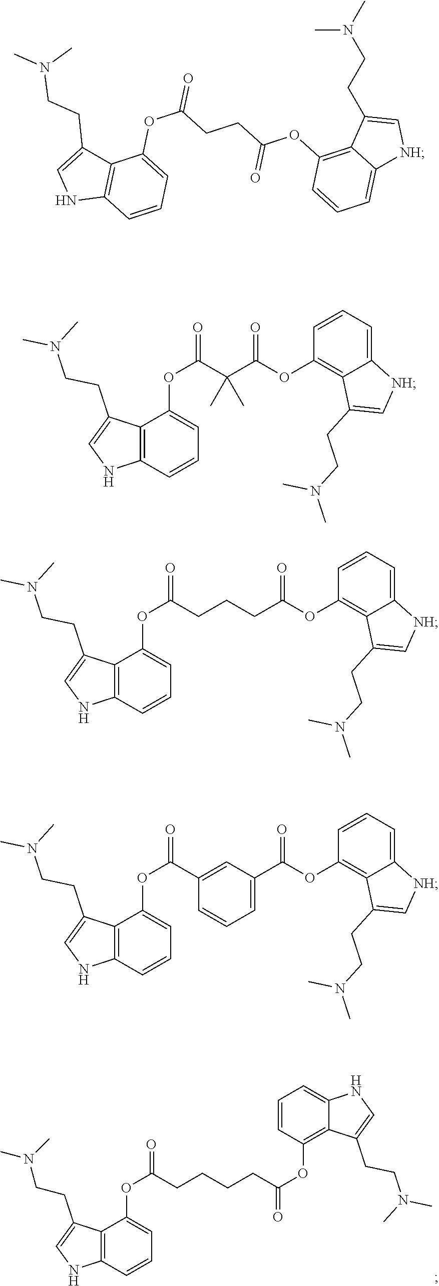

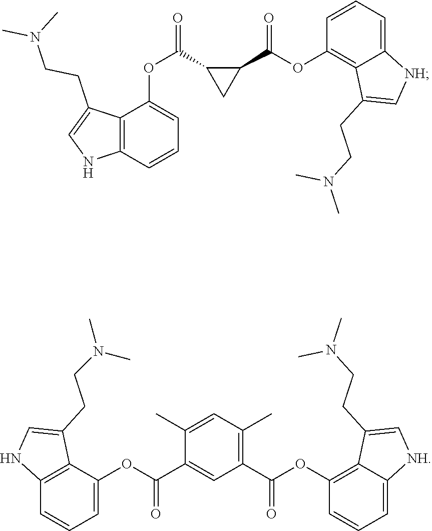 [SMILES: CN(C)CCc1c[nH]c2cccc(OC(=O)[C@H]3C[C@@H]3C(=O)Oc3cccc4[nH]cc(CCN(C)C)c34)c12 ; Cc1cc(C)c(C(=O)Oc2cccc3[nH]cc(CCN(C)C)c23)cc1C(=O)Oc1cccc2[nH]cc(CCN(C)C)c12]

### Derivatives

In embodiments of this aspect, a compound in accordance with the disclosure can comprise a derivative of the compounds. Some embodiments, for example, comprise a deuterated form of the compound, (i.e., one or more hydrogen atoms substituted with deuterium). In such embodiments, the deuterated forms of the compounds can exhibit an extended plasma half life and/or reduce the formation of certain metaoblites, relative to the non-deuterated version of the same compound(s). Deuterated forms of the compounds can comprise deuterium in an amount that is enriched for deuterium at least in one position of the structure that is above the natural abundance of deuterium (i.e., above about 0.015%). In embodiments a compound enriched for deuterium is from about 10% to over 95% enriched for deuterium at one or more positions in the structure. Certain deuterated tryptamine alkaloids (e.g., psilocin enriched for deuterium) are described in U.S. Pat. No. 11,000,534, the disclosure of which is incorporated herein by reference. Deuterated forms of compounds can be prepared using techniques generally known in the art such as, for example, using deuterated reactants/precursors, utilizing hydrogen-deuterium exchange reactions, and the like.

In some additional embodiments, a compound in accordance with the disclosure can comprise a halogenated form of the compound, i.e., one or more hydrogen atoms in the structure replaced by one or more of F, Cl, Br, and/or I. In such embodiments the compounds can exhibit an extended plasma half life and/or reduce the formation of certain metaoblites, relative to the non-halogenated version of the same compound(s). Halogenated forms of the compounds can comprise one or more halogens in an increased amount at least in one position of the structure that typically comprises hydrogen. In embodiments a halogenated compound includes from about 10% to over 95% substitution of a halogen at one or more positions in the structure. Certain halogenated tryptamine alkaloids (e.g., psilocin and psilocybin derivatives comprising halogens) have been described (e.g., Blair JB, et al. J Med Chem. 2000 Nov; 43(24):4701-4710; and Nichols, D.E. (2017). Chemistry and Structure—Activity Relationships of Psychedelics. In: Halberstadt, A.L., Vollenweider, F.X., Nichols, D.E. (eds) Behavioral Neurobiology of Psychedelic Drugs. Current Topics in Behavioral Neurosciences, vol 36. Springer, Berlin, Heidelberg, each of which are incorporated herein by reference). Halogenated forms of compounds can be prepared using techniques genereally known in the art such as, for example, using halogenated reactants/precursors, utilizing halogenation reactions (fluorination, chlorination, bromination, and/or iodination), and the like. In embodiments, the halogenated form of the compound comprises a halogen selected from F or Cl. In embodiments, the halogenated form of the compound comprises F.

In any of the above embodiments relating to deuterated and halogenated forms of the compounds, the compounds retain 5-HT receptor agonist activity. In some further embodiments relating to deuterated and halogenated forms of the compounds, the compounds retain 5-HT receptor selectivity. In yet further embodiments relating to deuterated and halogenated forms of the compounds, the compounds retain 5-HT receptor selectivity and agonist activity.

It will be appreciated that the compounds disclosed herein may comprise at least one stereogenic center in the structure. The chiral center(s) can be present in either the (R—) or (S—) (or alternatively, (D) or (L)) configuration as enantiomers or diastereomers, or combinations and mixtures thereof, any and all of which fall within the scope of the disclosure.

### Salts and Prodrugs

The compounds in accordance with the disclosure include pharmaceutically acceptable salts, esters, amides, and prodrugs thereof, including but not limited to carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S.M. et al., “Pharmaceutical Salts,” J. Pharm. Sci., 1977;66:1-19 which is incorporated herein by reference.)

Examples of pharmaceutically acceptable, non-toxic esters of the compounds of this invention include C₁-C₆ alkyl esters, wherein the alkyl group is a straight or branched, substituted or unsubstituted, C₅ -C₇ cycloalkyl esters, as well as arylalkyl esters such as benzyl and triphenylmethyl. C₁ -C₄ alkyl esters are preferred, such as methyl, ethyl, 2,2,2-trichloroethyl, and tert-butyl. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines, wherein the alkyl groups are straight or branched. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The term “prodrug” refers to compounds that are rapidly transformed in vivo to yield the parent compound of the above formulae, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

In embodiments, the compounds disclosed herein may exhibit the properties of a prodrug without any further structural modification (i.e., without addition of an ester or an amide functional group(s)). In such embodiments, the compounds may be hydrolysable (e.g., at the 4 position of the indole ring) under typical physiological conditions upon administration (e.g., in the bloodstream or gut, or converted in the liver to psilocin or an active derivative thereof).

### Methods, Uses, and Therapeutics Applications

As discussed herein, compounds in accordance with the disclosure can act as 5-HT receptor agonists, and thus, can exert a wide range of effects associated with various biological responses and processes that are generally known in the art (e.g., neurological and neuropsychiatric). For example, the compounds can act as hallucinogens, empathogens, antipsychotics, antidepressants, antiemetics, anorectics, and analgesics (nociceptive pathway inhibitors/antinociceptive agents), and can affect emotion and mood (e.g., anxiety and aggression), cognitive performance, sexual performance, pain perception, learning memory, and appetite among others.

Accordingly, in an aspect, the disclosure provides methods of treating one or more conditions that are responsive to serotonin receptor activation (i.e., 5-HT2_{A}, 5-HT2_{B}, 5-HT2_{C}), comprising the use or administration of the compounds described herein. The methods can comprise administering to a subject in need of such treatment an effective amount (i.e., therapeutically effective amount) of one or more compounds of the disclosure as described herein (i.e., compounds of Formulas (I) or (II)) or a pharmaceutical composition thereof.

In embodiments the methods can be used to treat a neurological disease, and comprise administering a compound of the disclosure to a patient in need of treatment. As referred to herein, a “neurological disease” refers to any condition or disease involving the nervous system, for example, diseases that involve the central nervous system (brain, brainstem and cerebellum), the peripheral nervous system (including cranial nerves), and the autonomic nervous system. Within the broad scope of neurological diseases, a “neurodegenerative disease” refers to a neurological disease marked by the loss of nerve cells or damage to nerve cells, including non-limiting examples of Alzheimer's disease, Parkinson's disease, amyotrophic lateral sclerosis, tauopathies (including frontotemporal dementia), Huntington's disease, and the like. Further non-limiting examples of neurological diseases include headache, stupor and coma, dementia, seizure, sleep disorders, trauma, infections, neoplasms, neuro-ophthalmological conditions, movement disorders, demyelinating diseases, spinal cord disorders, disorders of peripheral nerves, muscle and neuromuscular junctions, among others. Addiction, mental illness, and personality disorders are also examples of neurological diseases and include a broad scope of conditions such as those discussed herein and as generally known in the art.

In embodiments, the compounds are used in the treatment of pain (e.g., a painful condition) or a disease associated with pain. In embodiments, the use or method provides a form of pain management (e.g., reduce, eliminate, mitigate or relieve the symptoms). Non-liming examples of pain include neuropathic pain (e.g., peripheral neuropathic pain), central pain, deafferentation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. Any of the pain-related conditions can comprise one or more types of pain (e.g. nociceptive pain, inflammatory pain, neuropathic pain, etc.). In embodiments, a particular source or type pain can dominate.

In certain embodiments, compounds are used in the treatment of a psychiatric disorder. The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

Accordingly, in some particular embodiments the disclosure provides for the use of the compounds of the disclosure in the treatment of one or more conditions including: dependence, addiction, and/or abuse of substances including, for example, alcohol, tobacco, nicotine, stimulants, and drugs (e.g., cocaine, cannabis, opioids); treatment of anxiety disorders, for example, post-traumatic stress disorder (PTSD), generalized anxiety disorder (GAD), obsessive-compulsive disorder (OCD), advanced-stage cancer-related anxiety, psychological distress (i.e., associated with existential crisis of terminal disease), and adjustment disorder with anxiety; treatment of depression, for example, cancer-related depression, treatment-resistant depression, major depressive disorder, severe existential depression; treatment of suicidality (i.e., ideation and actual attempt); treatment of demoralization including demoralization in older, long-term AIDS survivor men (OLTAS); treatment of pain, for example, cluster headaches, chronic pain, intractable phantom pain, or a disease associated with pain; treatment of personality disorders, for example, dysfunctional social cognition, maladaptive narcissism, borderline personality disorder (BPD), narcissistic personality disorder (NPD), psychopathy, emotional dysregulation and domestic violence/violence against one's partner; treatment of epilepsy; treatment of inflammation; and treatment of neurological diseases.

In embodiments the disclosure provides a method for treating a depressive disorder in a subject in need thereof, wherein the method comprises administering to the subject a composition comprising a compound of Formula (I) or (II). In certain embodiments, the compound of Formula (I) is a compound of Formula (Ib). In certain embodiments, the compound of Formula (Ib) is a compound selected from 3-(2-(dimethylamino)ethyl)-1H-indol-4-yl 4-bromobenzoate; 3-(2-(dimethylamino)ethyl)-1H-indol-4-yl furan-3-carboxylate; 3-(2-(dimethylamino)ethyl)-1H-indol-4-yl 2-fluorobenzoate; 3-(2-(dimethylamino)ethyl)-1H-indol-4-yl 3-fluorobenzoate; 3-(2-(dimethylamino)ethyl)-1H-indol-4-yl 4-fluorobenzoate; or 3-(2-(dimethylamino)ethyl)-1H-indol-4-yl thiophene-2-carboxylate as described herein. In further embodiments, the compound of Formula (II) is a compound of Formula (IIb). In certain embodiments, the compound of Formula (IIb) is a compound selected from bis(3-(2-(dimethylamino)ethyl)-1H-indol-4-yl) isophthalate; bis(3-(2-(dimethylamino)ethyl)-1H-indol-4-yl) 4,6-dimethylisophthalate; bis(3-(2-(dimethylamino)ethyl)-1H-indol-4-yl) (1S,2S)-cyclopropane-1,2-dicarboxylate; bis(3-(2-(dimethylamino)ethyl)-1H-indol-4-yl) 2,2-dimethylmalonate; bis(3-(2-(dimethylamino)ethyl)-1H-indol-4-yl) succinate; bis(3-(2-(dimethylamino)ethyl)-1H-indol-4-yl) glutarate; or bis(3-(2-(dimethylamino)ethyl)-1H-indol-4-yl) adipate as described herein.

In another embodiment, the disclosure provides a method for treating a mood disorder in a subject in need thereof, wherein the method comprises administering to the subject a composition comprising a compound of Formula (I) or (II). In certain embodiments, the compound of Formula (I) is a compound of Formula (Ib). In certain embodiments, the compound of Formula (Ib) is a compound selected from 3-(2-(dimethylamino)ethyl)-1H-indol-4-yl 4-bromobenzoate; 3-(2-(dimethylamino)ethyl)-1H-indol-4-yl furan-3-carboxylate; 3-(2-(dimethylamino)ethyl)-1H-indol-4-yl 2-fluorobenzoate; 3-(2-(dimethylamino)ethyl)-1H-indol-4-yl 3-fluorobenzoate; 3-(2-(dimethylamino)ethyl)-1H-indol-4-yl 4-fluorobenzoate; or 3-(2-(dimethylamino)ethyl)-1H-indol-4-yl thiophene-2-carboxylate as described herein. In further embodiments, the compound of Formula (II) is a compound of Formula (IIb). In certain embodiments, the compound of Formula (IIb) is a compound selected from bis(3-(2-(dimethylamino)ethyl)-1H-indol-4-yl) isophthalate; bis(3-(2-(dimethylamino)ethyl)-1H-indol-4-yl) 4,6-dimethylisophthalate; bis(3-(2-(dimethylamino)ethyl)-1H-indol-4-yl) (1S,2S)-cyclopropane-1,2-dicarboxylate; bis(3-(2-(dimethylamino)ethyl)-1H-indol-4-yl) 2,2-dimethylmalonate; bis(3-(2-(dimethylamino)ethyl)-1H-indol-4-yl) succinate; bis(3-(2-(dimethylamino)ethyl)-1H-indol-4-yl) glutarate; or bis(3-(2-(dimethylamino)ethyl)-1H-indol-4-yl) adipate as described herein. In further embodiments, the mood disorder is psychological distress (e.g., depression or anxiety) related with a life-threatening disease.

In another embodiment, the disclosure provides a method for treating an anxiety disorder in a subject in need thereof, the method comprising administering to the subject a composition comprising a compound of Formula (I). In certain embodiments, the compound of Formula (I) is a compound of Formula (Ib). In certain embodiments, the compound of Formula (Ib) is a compound selected from 3-(2-(dimethylamino)ethyl)-1H-indol-4-yl 4-bromobenzoate; 3-(2-(dimethylamino)ethyl)-1H-indol-4-yl furan-3-carboxylate; 3-(2-(dimethylamino)ethyl)-1H-indol-4-yl 2-fluorobenzoate; 3-(2-(dimethylamino)ethyl)-1H-indol-4-yl 3-fluorobenzoate; 3-(2-(dimethylamino)ethyl)-1H-indol-4-yl 4-fluorobenzoate; or 3-(2-(dimethylamino)ethyl)-1H-indol-4-yl thiophene-2-carboxylate as described herein. In further embodiments, the compound of Formula (II) is a compound of Formula (IIb). In certain embodiments, the compound of Formula (IIb) is a compound selected from bis(3-(2-(dimethylamino)ethyl)-1H-indol-4-yl) isophthalate; bis(3-(2-(dimethylamino)ethyl)-1H-indol-4-yl) 4,6-dimethylisophthalate; bis(3-(2-(dimethylamino)ethyl)-1H-indol-4-yl) (1S,2S)-cyclopropane-1,2-dicarboxylate; bis(3-(2-(dimethylamino)ethyl)-1H-indol-4-yl) 2,2-dimethylmalonate; bis(3-(2-(dimethylamino)ethyl)-1H-indol-4-yl) succinate; bis(3-(2-(dimethylamino)ethyl)-1H-indol-4-yl) glutarate; or bis(3-(2-(dimethylamino)ethyl)-1H-indol-4-yl) adipate, as described herein.

In another embodiment, the dislcosure provides a method for treating an addiction disorder in a subject in need thereof, the method comprising administering to the subject a composition comprising a compound of Formula (I). In certain embodiments, the compound of Formula (I) is a compound of Formula (Ib). In certain embodiments, the compound of Formula (Ib) is a compound selected from 3-(2-(dimethylamino)ethyl)-1H-indol-4-yl 4-bromobenzoate; 3-(2-(dimethylamino)ethyl)-1H-indol-4-yl furan-3-carboxylate; 3-(2-(dimethylamino)ethyl)-1H-indol-4-yl 2-fluorobenzoate; 3-(2-(dimethylamino)ethyl)-1H-indol-4-yl 3-fluorobenzoate; 3-(2-(dimethylamino)ethyl)-1H-indol-4-yl 4-fluorobenzoate; or 3-(2-(dimethylamino)ethyl)-1H-indol-4-yl thiophene-2-carboxylate as described herein. In further embodiments, the compound of Formula (II) is a compound of Formula (IIb). In certain embodiments, the compound of Formula (IIb) is a compound selected from bis(3-(2-(dimethylamino)ethyl)-1H-indol-4-yl) isophthalate; bis(3-(2-(dimethylamino)ethyl)-1H-indol-4-yl) 4,6-dimethylisophthalate; bis(3-(2-(dimethylamino)ethyl)-1H-indol-4-yl) (1S,2S)-cyclopropane-1,2-dicarboxylate; bis(3-(2-(dimethylamino)ethyl)-1H-indol-4-yl) 2,2-dimethylmalonate; bis(3-(2-(dimethylamino)ethyl)-1H-indol-4-yl) succinate; bis(3-(2-(dimethylamino)ethyl)-1H-indol-4-yl) glutarate; or bis(3-(2-(dimethylamino)ethyl)-1H-indol-4-yl) adipate, as described herein.

In another embodiment, the disclosure provides a method for treating a pain disorder in a subject in need thereof, the method comprising administering to the subject a composition comprising a compound of Formula (I). In certain embodiments, the compound of Formula (I) is a compound of Formula (Ib). In certain embodiments, the compound of Formula (Ib) is a compound selected from 3-(2-(dimethylamino)ethyl)-1H-indol-4-yl 4-bromobenzoate; 3-(2-(dimethylamino)ethyl)-1H-indol-4-yl furan-3-carboxylate; 3-(2-(dimethylamino)ethyl)-1H-indol-4-yl 2-fluorobenzoate; 3-(2-(dimethylamino)ethyl)-1H-indol-4-yl 3-fluorobenzoate; 3-(2-(dimethylamino)ethyl)-1H-indol-4-yl 4-fluorobenzoate; or 3-(2-(dimethylamino)ethyl)-1H-indol-4-yl thiophene-2-carboxylate as described herein. In further embodiments, the compound of Formula (II) is a compound of Formula (IIb). In certain embodiments, the compound of Formula (IIb) is a compound selected from bis(3-(2-(dimethylamino)ethyl)-1H-indol-4-yl) isophthalate; bis(3-(2-(dimethylamino)ethyl)-1H-indol-4-yl) 4,6-dimethylisophthalate; bis(3-(2-(dimethylamino)ethyl)-1H-indol-4-yl) (1S,2S)-cyclopropane-1,2-dicarboxylate; bis(3-(2-(dimethylamino)ethyl)-1H-indol-4-yl) 2,2-dimethylmalonate; bis(3-(2-(dimethylamino)ethyl)-1H-indol-4-yl) succinate; bis(3-(2-(dimethylamino)ethyl)-1H-indol-4-yl) glutarate; or bis(3-(2-(dimethylamino)ethyl)-1H-indol-4-yl) adipate, as described herein. In further embodiments, the pain disorder is migraine, arthritis, headache, back pain, bursitis, chronic pain, acute pain, musculoskeletal pain, osteoarthritis, psoriatic arthritis, rheumatoid arthritis, or sciatica. In embodiments, the pain disorder is migraine. In embodiments, the pain disorder is arthritis. In embodiments, the pain disorder is headache. In embodiments, the pain disorder is back pain. In embodiments, the pain disorder is bursitis. In embodiments, the pain disorder is chronic pain. In embodiments, the pain disorder is acute pain. In embodiments, the pain disorder is musculoskeletal pain. In embodiments, the pain disorder is osteoarthritis. In embodiments, the pain disorder is psoriatic arthritis. In embodiments, the pain disorder is rheumatoid arthritis. In embodiments, the pain disorder is sciatica. In embodiments, the pain disorder is migraine or headache.

In another embodiment, the disclosure provides a method for treating a psychiatric disorder in a subject in need thereof, the method comprising administering to the subject a composition comprising a compound of Formula (I). In certain embodiments, the compound of Formula (I) is a compound of Formula (Ib). In certain embodiments, the compound of Formula (Ib) is a compound selected from 3-(2-(dimethylamino)ethyl)-1H-indol-4-yl 4-bromobenzoate; 3-(2-(dimethylamino)ethyl)-1H-indol-4-yl furan-3-carboxylate; 3-(2-(dimethylamino)ethyl)-1H-indol-4-yl 2-fluorobenzoate; 3-(2-(dimethylamino)ethyl)-1H-indol-4-yl 3-fluorobenzoate; 3-(2-(dimethylamino)ethyl)-1H-indol-4-yl 4-fluorobenzoate; or 3-(2-(dimethylamino)ethyl)-1H-indol-4-yl thiophene-2-carboxylate as described herein. In further embodiments, the compound of Formula (II) is a compound of Formula (IIb). In certain embodiments, the compound of Formula (IIb) is a compound selected from bis(3-(2-(dimethylamino)ethyl)-1H-indol-4-yl) isophthalate; bis(3-(2-(dimethylamino)ethyl)-1H-indol-4-yl) 4,6-dimethylisophthalate; bis(3-(2-(dimethylamino)ethyl)-1H-indol-4-yl) (1S,2S)-cyclopropane-1,2-dicarboxylate; bis(3-(2-(dimethylamino)ethyl)-1H-indol-4-yl) 2,2-dimethylmalonate; bis(3-(2-(dimethylamino)ethyl)-1H-indol-4-yl) succinate; bis(3-(2-(dimethylamino)ethyl)-1H-indol-4-yl) glutarate; or bis(3-(2-(dimethylamino)ethyl)-1H-indol-4-yl) adipate, as described herein. In embodiments, the neurological or psychiatric disorder is narcolepsy, Alzheimer's disease, attention deficit hyperactivity disorder (ADHD), schizophrenia, Parkinson's disease, or depression. In embodiments, the neurological or psychiatric disorder is attention deficit hyperactivity disorder (ADHD). In embodiments, the neurological or psychiatric disorder is schizophrenia. In embodiments, the neurological or psychiatric disorder is Parkinson's disease. In embodiments, the neurological or psychiatric disorder is depression.

The compounds and compositions of the disclosure as described herein may also be administered in combination with one or more secondary therapeutic agents. Thus, in certain embodiment, the method also includes administering to a subject in need of such treatment an effective amount of one or more compounds of the disclosure as described herein (i.e., compounds of formula (I) or (II) or a pharmaceutical composition of the disclosure as described herein and one or more secondary therapeutic agents. Examples of suitable secondary therapeutic agents include, but are not limited to, anti-depressants, cannabinoids, stimulants, anti-inflammatory agents, steroids, barbiturates, analgesics, sleep aid/agents (e.g. melatonin or eszopiclone), anxiolytics, antipsychotics, antinociceptive agents, NSAIDs, mood enhancing agents, and the like or a combination thereof. When administered as a combination, the compounds and compositions of the disclosure and the additional therapeutic agents can be formulated as separate compositions that are given simultaneously or sequentially, or the therapeutic agents can be given as a single composition. In certain embodiments, the secondary therapeutic agent may be administered in an amount below its established half maximal inhibitory concentration (IC₅₀). For example, the secondary therapeutic agent may be administered in an amount less than 1% of, e.g., less than 10%, or less than 25%, or less than 50%, or less than 75%, or even less than 90% of the inhibitory concentration (IC₅₀).

In certain embodiments, the method further comprises administering to the subject in need thereof an additional therapy. In embodiments, the additional therapy can comprise any form of therapy that may be effective to generate a therapeutic response including, for example, counseling (e.g., mental health counseling, addiction counseling, behavioral therapy such as cognitive behavioral therapy (CBT), and the like), as well as pharmaceutical agents that may be useful in the treatment of one or more underlying conditions or diseases that may cause or exacerbate the condition(s) being treatment by the disclosed methods.

In any of the above aspects and embodiments relating to uses and methods of treatment, a combination treatment may show a synergistic effect relative to the treatments administered as individual therapies.

### Pharmaceutical Compositions

In some aspects, the disclosure provides pharmaceutical compositions comprising a compound as described herein, and at least one pharmaceutically acceptable carrier, solvent, adjuvant or diluent.

For administration as compositions, including pharmaceutical compositions, the compounds are ordinarily combined with one or more carriers, diluents, and/or adjuvants appropriate for the indicated route of administration. The compounds may be mixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compounds disclosed herein can be administered as the sole active pharmaceutical agent, or they can be used in combination with one or more other compounds useful for carrying out the methods and uses. When used or administered as a combination, the therapeutic agents can be formulated as separate compositions that are given at the same time or different times, or the therapeutic agents can be given as a single composition.

The compounds can be prepared in a solid form (including granules, powders or suppositories) or in a liquid form (e.g., solutions, suspensions, or emulsions). The disclosed compounds may be applied in a variety of solutions and may be subjected to conventional pharmaceutical operations such as sterilization and/or may contain conventional adjuvants, such as preservatives, stabilizers, wetting agents, emulsifiers, buffers etc.

The disclosed compounds may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. One or more compounds in accordance with the disclosure may be present in association with one or more non-toxic pharmaceutically acceptable carriers and/or diluents and/or adjuvants, and if desired other active ingredients. Such pharmaceutical compositions may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In embodiments, such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions in accordance with the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds and pharmaceutical compositions in accordance with the disclosure may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds and pharmaceutical compositions in accordance with the disclosure may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The amount of compound(s) administered will depend upon a variety of factors, including, for example, the particular indication being treated, the mode of administration, whether the desired benefit is prophylactic or therapeutic, the severity of the indication being treated, the age and weight of the patient, the bioavailability of the particular compound(s), the metabolism rate and efficiency of the compound under the selected route of administration, etc. Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an ICso of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Compositions in accordance with the disclosure can include an amount of the compounds disclosed herein over a wide range, for example, from about 0.01 mg/mL to about 50 mg/mL, or from about 0.5 mg/mL to about 25 mg/mL, from about 0.1 mg/mL to about 10 mg/mL, or from about 0.1 mg/mL to about 5 mg/mL, or from about 0.1 mg/mL to about 1 mg/mL.

As mentioned above, compositions in accordance with the disclosure can be administered in dosages based on the weight of a subject, and are useful in the treatment of the indications and conditions described herein. In embodiments, the composition is a pharmaceutical composition and comprises an effective amount of about 0.05 mg/kg to about 2.0 mg/kg of the compound of Formula (I) or (II), or a pharmaceutically acceptable salt, prodrug, hydrate, or solvate thereof. In another embodiment the pharmaceutical composition comprises an effective amount of about 0.1 mg/kg to about 1.0 mg/kg of the compound of Formula (I) or (II) or a pharmaceutically acceptable salt, prodrug, hydrate, or solvate thereof. In another embodiment the pharmaceutical composition comprises about 0.2 mg/kg to about 0.6 mg/kg of the compound of Formula (I) or (II) or a pharmaceutically acceptable salt, prodrug, hydrate, or solvate thereof. In another embodiment the pharmaceutical composition comprises about 0.3 mg/kg to about 0.5 mg/kg of the compound of Formula (I) or (II) or a pharmaceutically acceptable salt, prodrug, hydrate, or solvate thereof. The amount of active compound(s) that may be combined with the carrier materials to produce a single dosage form will vary depending upon the subject to be treated and the particular mode of administration. Dosage unit forms will generally contain between from about 1 mg to about 500 mg of an active compound/ingredient per kilogram of body weight per day. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

The compound(s) described herein, or compositions thereof, will generally be used in an amount effective to achieve the intended result, for example in an amount effective to treat or prevent the particular disease being treated. By therapeutic benefit is meant eradication, delaying onset or progression, or amelioration of the underlying disorder being treated and/or eradication, delaying onset or progression, or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

### Screening Compounds for Activity

Compounds in accordance with the disclosure generally exhibit 5-HT₂ receptor agonist activity. Screening the compounds for activity as 5-HT receptor agonists can be accomplished using any assay described herein, and/or as known in the art, including commercially available assays. In particular embodiments, the compounds exhibit agonist activity for one or more of the three G_{q/11} protein-coupled receptor subtypes, 5-HT_{2A}, 5-HT_{2B}, and/or 5-HT_{2C}. In some preferred embodiments, the compounds exhibit agonist activity for one or both of the 5-HT 2A and/or 5-HT_{2C}receptors. In yet other preferred embodiments, the compounds exhibit agonist activity for 5-HT_{2A}. In yet other preferred embodiments, the compounds exhibit agonist activity for 5-HT_{2C}. In some preferred embodiments, the compounds exhibit agonist activity for one or both of the 5-HT_{2A} or 5-HT_{2C} receptors, and do not exhibit agonist activity for the 5-HT_{2B} receptor. In some other preferred embodiments, the compounds exhibit agonist activity for 5-HT_{2C} and do not exhibit agonist activity for 5-HT_{2B}. In some other preferred embodiments, the compounds exhibit agonist activity for 5-HT_{2A} and do not exhibit agonist activity for 5-HT_{2B}.

Thus, in certain embodiments, the compounds described herein can exhibit agonist activity for 5-HT₂ receptors and/or selectivity for one or more of the receptor subtypes 5-HT_{2A}, 5-HT_{2B}, and 5-HT_{2C}. The compounds can be screened and selected for 5-HT receptor agonist activity using any of the assays described herein or as are known in the art including, for example, assays that monitor or characterize one or more of the canonical and/or non-canonical G protein signaling pathway. Non-limiting examples of G protein signaling pathway assays include, assays that measure G protein recruitment/activation, (e.g., by release of cyclic adenosine, inositol phosphate accumulation/hydrolysis (or PLC activation), and/or Ca²⁺ mobilization), assays that monitor arachidonic acid release, assays that monitor β-arrestin recruitment or signaling, and assays that monitor 5-HT receptor conformational changes.

In embodiments, the compounds in accordance with the disclosure can be screened to determine binding affinity, including binding specificity, for one or more of the receptors 5-HT_{2A}, 5-HT_{2B}, and/or 5-HT_{2C} using binding assays such as those described herein and/or as generally known in the art (e.g., competitive binding assays, ligand displacement assays, etc.). In example embodiments, the binding affinity for a compound to one or more of the 5-HT_{2A}, 5-HT_{2B}, and/or 5-HT_{2C} receptors can be determined by an assay that measures the displacement of one or more labelled ligands (e.g., radioligands), including antagonist and/or agonist ligands, which can reflect binding to either or both active and inactive receptor conformations and determine binding constants.

### Methods of Preparation

A number of references provide commonly known chemical synthetic schemes and conditions are useful for synthesizing the disclosed compounds (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, 4^{th} Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. In embodiments the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/I, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art. In embodiments protecting groups can be selected from 9-fluorenylmethoxycarbonyl (Fmoc), p-nitrobenzenesulfonyl, t-butyldimethylsilyl (TBS), or other protecting groups known in the art.

Thus, the compounds disclosed herein can be made using procedures familiar to a person skilled in the art in addition to, or in combination with, those procedures as described below. For example, compounds of Formula (I) or (II) or intermediate compounds as described herein, can be prepared according to general procedures (below), and/or analogous synthetic procedures. One of skill in the art can adapt any of the general or specific reaction schemes described below to fit the desired target molecule. In certain adaptations of the synthetic reactions/schemes, one of skill in the art will use different reagents to affect one or more of the individual steps or to use protected versions of certain of the substituents. Additionally, one skilled in the art would recognize that compounds of the disclosure can be synthesized using different routes altogether.

### Preparation of Intermediates for Generating Compounds of Formula (I) and (II).

It will be appreciated that one or more intermediate compounds can be used in the preparation of the compounds disclosed herein. For example, In embodiments intermediate compounds comprising derivatives of 4-hydroxy-N,N-dimethyltryptamine (psilocin) such as 4-benzyloxypsilocin (O-Bn psilocin; (A-1)) and N-t-butyldimethylsilylpsilocin (N-TBS psilocin (A-2)) can be obtained from a commercial source or prepared according to the reactions depicted below.

Various intermediate compounds can be made using procedures familiar to the person of ordinary skill in the art in addition to or in combination with those procedures as described herein. For example, compounds according to Formula (A-1) and (A-2), or derivatives thereof, can be prepared according to general synthetic procedures illustrated herein, and/or analogous synthetic procedures as generally known in the art.

For example, In embodiments, intermediates of Formula A-1 and A-2 can be prepared according to the following Scheme 1A.

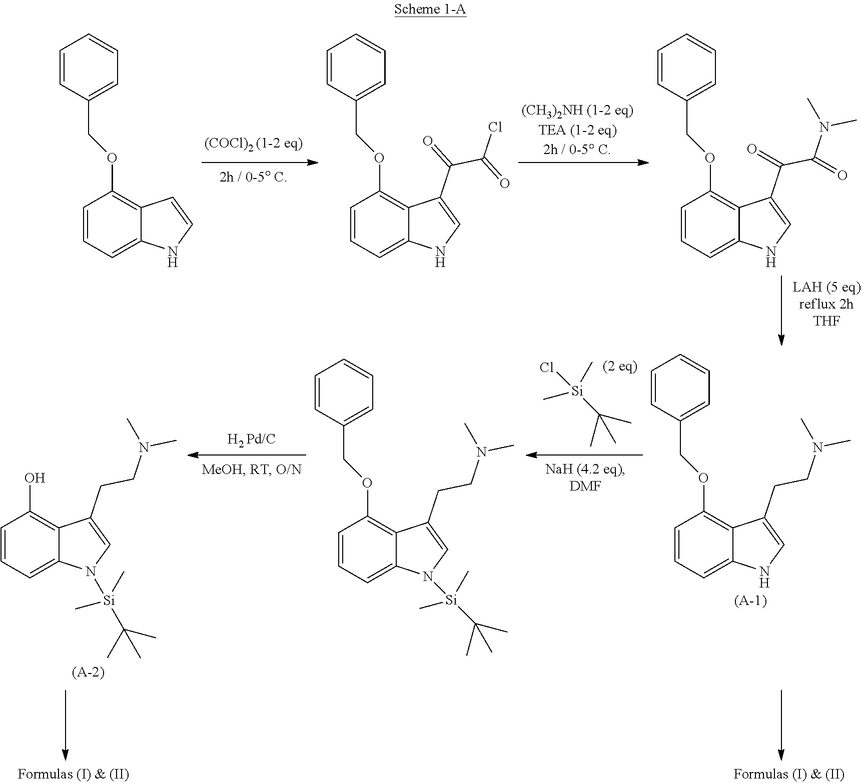

One of ordinary skill in the art can adapt any of the reaction schemes described herein, including the specific illustrative embodiments of the Examples, in order to generate compounds in accordance with the aspects and embodiments of the disclosure.

### NUMBERED EMBODIMENTS

In addition to the disclosure above, the Examples below, and the appended claims, the disclosure sets for the following numbered embodiments.
1. A compound, or derivative thereof, of Formula (I):

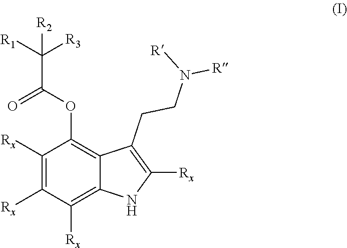

wherein
R₁ is absent or comprises hydrogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R₂ and R₃ independently comprise hydrogen, deuterium, halogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl; or
R₂ and R₃ together with the carbon atom to which they are attached form a C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl; and
each Rₓ is independently H, —OH, methyl, methoxy, C₁-C₃ alkyl, C₁-C₆ haloalkyl, deuterated C₁-C₃ alkyl, C₁-C₃ alkoxy, —C(═O)OH, —C(═O)NR₂R₃, or halogen.
1a. The compound of embodiment 1, wherein R₁ is absent or comprises hydrogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R₂ and R₃ independently comprise hydrogen, deuterium, halogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl; or
R₂ and R₃ together with the carbon atom to which they are attached form a C₃-C₂₀ cycloalkyl, aryl, or heteroaryl, any of which may be optionally substituted with one or more halogen, C₁-C₆ alkyl, C₂-C₆ alkenyl, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl;
each Rₓ is independently H, —OH, methyl, methoxy, C₁-C₃ alkyl, C₁-C₆ haloalkyl, deuterated C₁-C₃ alkyl, C₁-C₃ alkoxy, —C(═O)OH, —C(═O)NR₂R₃, or halogen; and
wherein the compound is not

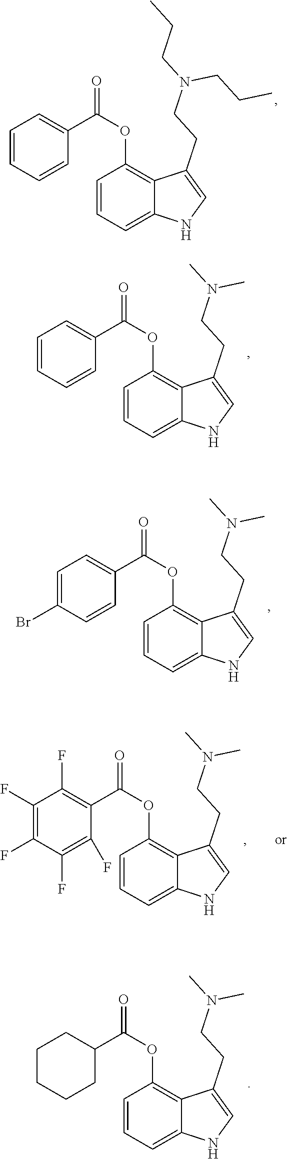

2. The compound of embodiment 1 or 1a, wherein R′ and R″ are independently a C₁-C₆ alkyl.
3. The compound of any of embodiments 1-2, wherein each Rₓ is independently H, —OH, methyl, methoxy, or halogen.
4. The compound of any of embodiments 1-3, wherein R₁ is absent and R₂ and R₃ with the carbon atom to which they are attached form a C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl.
5. The compound of any one of embodiments 1-4, wherein
   (i) one of R₂ or R₃ comprises hydrogen, and the other of R₂ or R₃ comprises C₂-C₆ alkenyl, C₂-C₆ alkynyl, C₁-C₆ alkoxy, or C₃-C₁₂ cycloalkyl; or
   (ii) R₂ and R₃ with the carbon atom to which they are attached form a C₃-C₁₂ cycloalkyl or C₃-C₁₂ heterocyclyl that is optionally substituted.
6. The compound of any one of embodiments 1-5, wherein the compound is a derivative comprising one or more deuterium substitutions for hydrogen.
7. The compound of any one of embodiments 1-5, comprising a structure according to Formula (Ib):

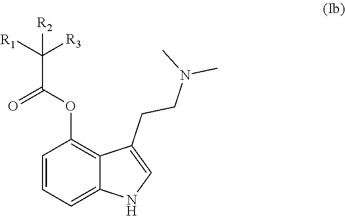

or pharmaceutically acceptable salt, ester, amide, and prodrug thereof, wherein
   R₁ is absent and R₂ and R₃ with the carbon atom to which they are attached form a C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl.
8. A compound comprising:
3-(2-(dimethylamino)ethyl)-1H-indol-4-yl 4-bromobenzoate;
3-(2-(dimethylamino)ethyl)-1H-indol-4-yl furan-3-carboxylate;
3-(2-(dimethylamino)ethyl)-1H-indol-4-yl 2-fluorobenzoate;
3-(2-(dimethylamino)ethyl)-1H-indol-4-yl 3-fluorobenzoate;
3-(2-(dimethylamino)ethyl)-1H-indol-4-yl 4-fluorobenzoate; or
3-(2-(dimethylamino)ethyl)-1H-indol-4-yl thiophene-2-carboxylate,
   or pharmaceutically acceptable salt, ester, amide, and prodrug thereof.
   9. A pharmaceutical composition, comprising the compound of any of embodiments 1-8 and a pharmaceutically acceptable carrier.
   10. A method for treating one or more conditions that are responsive to serotonin receptor activation, comprising administering to a subject in need thereof an effective amount of the compound of any of embodiments 1-8 or the pharmaceutical composition of embodiment 9.
   11. A method for treating a neurological disease, comprising administering to a subject in need thereof an effective amount of the compound of any of embodiments 1-8 or the pharmaceutical composition of embodiment 9.
   12. The method of embodiment 11, wherein the neurological disease is a neurodegenerative disease, stupor and coma, dementia, seizure, sleep disorder, trauma, infection, neoplasm, neuro-ophthalmological condition, movement disorder, demyelinating disease, spinal cord disorder, disorder of peripheral nerves, muscle and neuromuscular junctions, psychiatric disorder, pain, or a disease associated with pain.
   13. The method according to embodiment 12, wherein the psychiatric disorder is: an anxiety disorder including acute stress disorder agoraphobia, generalized anxiety disorder, obsessive-compulsive disorder, panic disorder, posttraumatic stress disorder, separation anxiety disorder, social phobia, or specific phobia; a childhood disorder including attention-deficit/hyperactivity disorder, conduct disorder, or oppositional defiant disorder; an eating disorder including anorexia nervosa or bulimia nervosa; a mood disorder including depression, bipolar disorder, cyclothymic disorder, dysthymic disorder, or major depressive disorder; a personality disorder including antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, or schizotypal personality disorder; a psychotic disorder including brief psychotic disorder, delusional disorder, schizoaffective disorder, schizophreniform disorder, schizophrenia, or shared psychotic disorder; a substance-related disorder including alcohol dependence, amphetamine dependence, cannabis dependence, cocaine dependence, hallucinogen dependence, inhalant dependence, nicotine dependence, opioid dependence, phencyclidine dependence, or sedative dependence; an adjustment disorder, autism, delirium, dementia, multi-infarct dementia, a learning or memory disorder including amnesia or age-related memory loss; or Tourette's disorder.
   14. The method of embodiment 12, wherein the neurological disease is pain, or a disease associated with pain.
   15. A compound, or derivative thereof, of Formula (II):

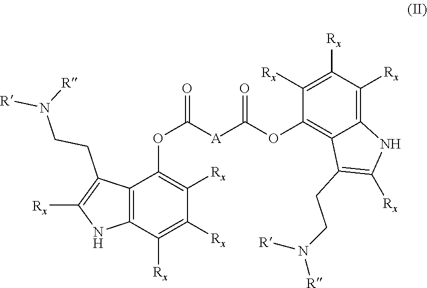

A is C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which may be optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R′ and R″ are independently H, C₁-C₆ alkyl, deuterated C₁-C₆ alkyl, C₂-C₆ alkenyl, C₂-C₆ alkynyl, or together with the N to which they are attached form a C₃-C₁₂ heterocyclyl or heteroaryl;
each Rₓ is independently H, —OH, methyl, methoxy, C₁-C₃ alkyl, C₁-C₆ haloalkyl, deuterated C₁-C₃ alkyl, C₁-C₃ alkoxy, —C(═O)OH, —C(═O)NR₂R₃, or halogen.
16. The compound of embodiment 15, wherein R′ and R″ are independently a C₁-C₆ alkyl.
17. The compound of any of embodiments 15-16, wherein each Rₓ is independently H, —OH, methyl, methoxy, or halogen.
18. The compound of any of embodiments 15-17, wherein A is C₁-C₆ alkyl, C₂-C₆ alkenyl, C₂-C₆ alkynyl, C₁-C₆ alkoxy, or C₃-C₁₂ cycloalkyl.
19. The compound of any one of embodiments 15-18, wherein the compound is a derivative comprising deuterium.
20. The compound of any one of embodiments 15-18, comprising a structure according to Formula (IIb):

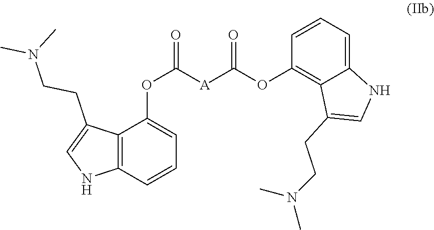

or pharmaceutically acceptable salt, ester, amide, and prodrug thereof, wherein
   A is C₁-C₆ alkyl, C₂-C₆ alkenyl, C₂-C₆ alkynyl, or C₁-C₆ alkoxy, C₃-C₁₂ cycloalkyl, C₃-C₁₂ heterocyclyl, aryl, or heteroaryl.
21. A compound comprising:
bis(3-(2-(dimethylamino)ethyl)-1H-indol-4-yl) isophthalate;
bis(3-(2-(dimethylamino)ethyl)-1H-indol-4-yl) 4,6-dimethylisophthalate;
bis(3-(2-(dimethylamino)ethyl)-1H-indol-4-yl) (1S,2S)-cyclopropane-1,2-dicarboxylate;
bis(3-(2-(dimethylamino)ethyl)-1H-indol-4-yl) 2,2-dimethylmalonate;
bis(3-(2-(dimethylamino)ethyl)-1H-indol-4-yl) succinate;
bis(3-(2-(dimethylamino)ethyl)-1H-indol-4-yl) glutarate; or
bis(3-(2-(dimethylamino)ethyl)-1H-indol-4-yl) adipate,
   or pharmaceutically acceptable salt, ester, amide, and prodrug thereof.
   22. A pharmaceutical composition, comprising the compound of any of embodiments 15-21 and a pharmaceutically acceptable carrier.
   23. A method for treating one or more conditions that are responsive to serotonin receptor activation, comprising administering to a subject in need thereof an effective amount of the compound of any of embodiments 15-21 or the pharmaceutical composition of embodiment 22.
   24. A method for treating a neurological disease, comprising administering to a subject in need thereof an effective amount of the compound of any of embodiments 15-21 or the pharmaceutical composition of embodiment 22.
   25. The method of embodiment 24, wherein the neurological disease is a neurodegenerative disease, stupor and coma, dementia, seizure, sleep disorder, trauma, infection, neoplasm, neuro-ophthalmological condition, movement disorder, demyelinating disease, spinal cord disorder, disorder of peripheral nerves, muscle and neuromuscular junctions, psychiatric disorder, pain, or a disease associated with pain.
   26. The method of embodiment 25, wherein the psychiatric disorder is: an anxiety disorder including acute stress disorder agoraphobia, generalized anxiety disorder, obsessive-compulsive disorder, panic disorder, posttraumatic stress disorder, separation anxiety disorder, social phobia, or specific phobia; a childhood disorder including attention-deficit/hyperactivity disorder, conduct disorder, or oppositional defiant disorder; an eating disorder including anorexia nervosa or bulimia nervosa; a mood disorder including depression, bipolar disorder, cyclothymic disorder, dysthymic disorder, or major depressive disorder; a personality disorder including antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, or schizotypal personality disorder; a psychotic disorder including brief psychotic disorder, delusional disorder, schizoaffective disorder, schizophreniform disorder, schizophrenia, or shared psychotic disorder; a substance-related disorder including alcohol dependence, amphetamine dependence, cannabis dependence, cocaine dependence, hallucinogen dependence, inhalant dependence, nicotine dependence, opioid dependence, phencyclidine dependence, or sedative dependence; an adjustment disorder, autism, delirium, dementia, multi-infarct dementia, a learning or memory disorder including amnesia or age-related memory loss; or Tourette's disorder.
   27. The method of embodiment 25, wherein the neurological disease is pain, or a disease associated with pain.

### EXAMPLES

The preparation of the compounds of the disclosure (e.g., of Formula (I) and Formula (II)) is illustrated by the following examples, which are not to be construed as limiting the disclosure in scope or spirit to the specific procedures and compounds described in them.

### General Experimental

All reagents were commercially available and used “as is” without further purification or drying. NMR spectra were obtained on a Bruker 400 MHz AVANCE™ III instrument. HPLC purifications were performed on Waters 600 Controller instrument utilizinga mixtures of ACN (0.1% FA) and H2O (0.1% FA) as eluent. MS/LC/MS analyses were performed on an Agilent 1100 series using an eclipse plus C18, 3.5 μm column. The standard gradient used was 5-100% ACN (0.1% FA) in H2O (0.1% FA).

### Example 1: Preparation of Ester Psilocin Derivatives of Formulas I and II

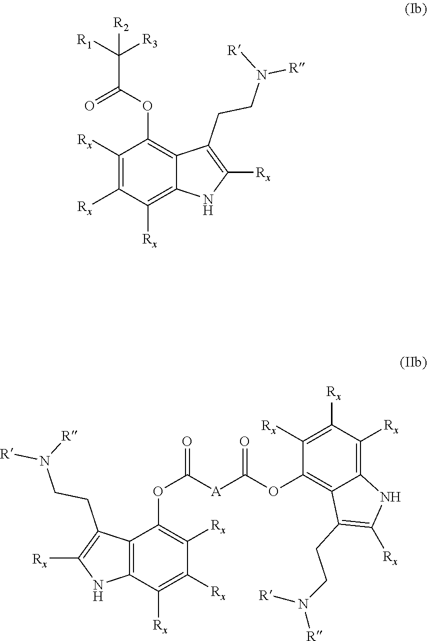

### Generalized Reaction Scheme

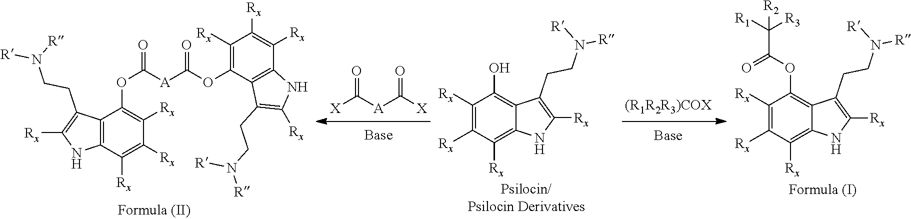

### Illustrative Reaction Scheme

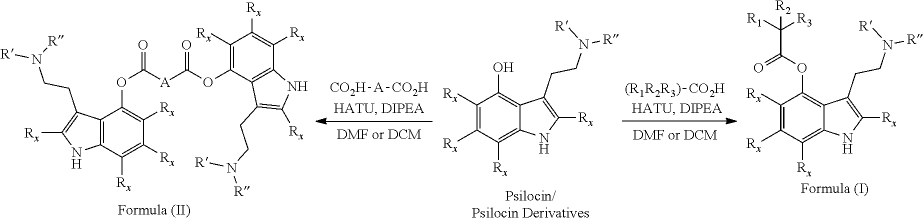

A series of ester psilocin derivatives are prepared according to the general reaction scheme above. Briefly, a solution comprising an amount of psiolicin or a psilocin derivative is prepared and reacted with an amount (e.g., a molar equivalent or slight excess) of a functionalized carboxylic acid or di-carboxylic acid along with 1-[Bis(dimethylamino)methylene]-1H-1,2,3-triazolo[4,5-b]pyridinium 3-oxid hexafluorophosphate (HATU), and N,N-diisopropylethylamine (DIPEA) in a suitable solvent (e.g., dimethylformamide (DMF) or dichloromethane (DCM)) to generate the corresponding ester derivative.

### General Procedure for Example 1

Under an inert atmosphere of argon gas, psilocin or a psilocin derivative (1-2 eq.) and functionalized dicarboxylic acid (1-1.4 eq.) were dissolved in anhydrous DMF (0.1-0.25 M) or DCM (0.1-0.25 M). The resulting mixture was cooled and stirred at 0° C. before HATU (1.2-2.1 eq.) and DI PEA (3-4 eq.) were added, respectively. The reaction was warmed to and maintained at a temperature of 25° C. for 2-24 hours. The reaction mixture was diluted with ethyl acetate (50 mL) upon completion. The organic phase was washed with brine (20 mL×3), it was dried over sodium sulfate, and the solvent was evaporated off. The crude reaction mixture was purified by reverse phase column chromatography, running a mobile phase of 90% to 60% H₂O (0.1% FA) in ACN (0.1% FA), and the product containing fractions lyophilized to afford the desired product.

### 1.A. 3-(2-(dimethylamino)ethyl)-1H-indol-4-yl 4-bromobenzoate

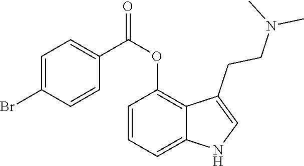 [SMILES: CN(C)CCc1c[nH]c2cccc(OC(=O)c3ccc(Br)cc3)c12]

The title compound, 3-(2-(dimethylamino)ethyl)-1H-indol-4-yl 4-bromobenzoate, was prepared according to the protocol described in general procedure for Example 1 using DMF as solvent and isolated as white solid (0.020 g, 10% yield). ¹H NMR (400 MHz, CD₃OD) δ 8.16 (d, J=8.6 Hz, 2H), 7.78 (d, J=8.6 Hz, 2H), 7.32 (d, J=8.1 Hz, 1H), 7.20 (s, 1H), 7.18-7.07 (m, 1H), 6.82 (d, J=7.5 Hz, 1H), 3.23 (t, J=7.7 Hz, 2H), 3.03 (t, J=7.7 Hz, 2H), 2.60 (s, 6H).

MS: measured m/z 387.07 [M+H]⁺. Purity by HPLC: 98.1% at 254 nm.

### 1.B. 3-(2-(dimethylamino)ethyl)-1H-indol-4-yl furan-3-carboxylate

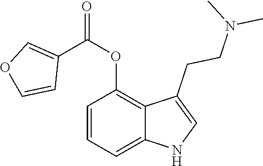 [SMILES: CN(C)CCc1c[nH]c2cccc(OC(=O)c3ccoc3)c12]

The title compound, 3-(2-(dimethylamino)ethyl)-1H-indol-4-yl furan-3-carboxylate, was prepared according to the general procedure for Example 1 using DMF as solvent and isolated as white solid (0.011 g, 16% yield). ¹H NMR (400 MHz, CD₃OD) δ 8.56 (dd, J=1.6, 0.8 Hz, 1H), 8.41 (s, 1H), 7.21-7.13 (m, 1H), 7.34 (dd, J=8.3, 0.8 Hz, 1H), 7.26 (s, 1H), 7.17 (t, J=7.9 Hz, 1H), 7.01 (dd, J=1.9, 0.8 Hz, 1H), 6.83 (dd, J=7.6, 0.8 Hz, 1H), 3.41-3.34 (m, 2H), 3.13 (t, J=7.6 Hz, 2H), 2.78 (s, 6H). MS: measured m/z 299.07 [M+H]⁺. Purity by HPLC: 95.9% at 254 nm.

### 1.C. 3-(2-(dimethylamino)ethyl)-1H-indol-4-yl2-fluorobenzoate

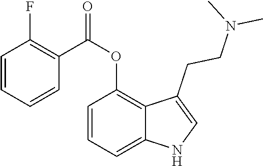 [SMILES: CN(C)CCc1c[nH]c2cccc(OC(=O)c3ccccc3F)c12]

The title compound, 3-(2-(dimethylamino)ethyl)-1H-indol-4-yl2-fluorobenzoate, was prepared according to the protocol described in the general procedure for Example 1 and isolated as white solid (0.046 g, 14% yield). ¹H NMR (400 MHz, CD₃OD) δ 8.30 (s, 1H), 8.05 (m, 1H), 7.65-7.55 (m, 1H), 7.29 — 7.15 (m, 3H), 7.08 (s, 1H), 7.01 (t, J=8.0 Hz, 1H), 6.70 (dd, J=7.6, 0.8 Hz, 1H), 3.13 (m, 2H), 2.94 (t, J=7.7 Hz, 2H), 2.52 (s, 6H). ¹⁹F NMR (376 MHz, CD₃OD) δ−110.17. MS: measured m/z 327.07 [M+H]⁺. Purity by HPLC: 98.8% at 254 nm.

### 1.D. 3-(2-(dimethvlamino)ethvI)-1H-indol-4-yl3-fluorobenzoate

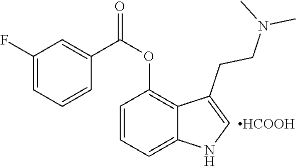

The title compound, 3-(2-(dimethylamino)ethyl)-1H-indol-4-yl3-fluorobenzoate, was prepared according to the protocol described in general procedure B using DCM as solvent and isolated in solid form as a formate salt, off-white in color (0.020 g, 5% yield). ¹H NMR (400 MHz, CDCl₃) δ 8.62 (s, 1H), 8.46 (s, 1H), 8.13-8.05 (m, 1H), 8.00-7.93 (m, 1H), 7.59-7.52 (m, 1H), 7.44-7.36 (m, 1H), 7.33-7.28 (m, 1H), 7.24-7.17 (m, 1H), 7.03 (s, 1H), 6.93-6.87 (m, 1H), 3.08-2.99 (m, 2H), 2.99-2.89 (m, 2H), 2.32 (s, 6H). ¹⁹F NMR (376 MHz, CDCl₃) δ−111.03. MS: measured m/z 327.13 [M−HCOO⁻]⁺. Purity by HPLC: 99.9% at 254 nm

### 1.E. bis(3-(2-(dimethylamino)ethyl)-1H-indol-4-yl) isophthalate

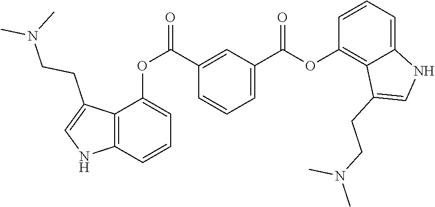 [SMILES: CN(C)CCc1c[nH]c2cccc(OC(=O)c3cccc(C(=O)Oc4cccc5[nH]cc(CCN(C)C)c45)c3)c12]

The title compound, bis(3-(2-(dimethylamino)ethyl)-1H-indol-4-yl) isophthalate, was prepared according to the protocol described in the general procedure for Example 1 using DMF as a solvent and isolated as a white powder (0.069 g, 52% yield). ¹H NMR (400 MHz, DMSO-d6) δ 11.12 (s, 2H), 8.92 (s, 1H), 8.57 (dd, J=7.8, 1.8 Hz, 2H), 7.95-7.86 (m, 1H), 7.31 (d, J=8.1 Hz, 2H), 7.18 (d, J=2.3 Hz, 2H), 7.14-7.03 (m, 2H), 6.84 (d, J=7.6 Hz, 2H), 2.74-2.66 (m, 4H), 2.43-2.35 (m, 4H), 1.93 (s, 12H). ESI-MS: measured m/z 539.2 [M+H]⁺. Purity by HPLC: 99.2% at 254 nm.

### 1.F. bis(3-(2-(dimethylamino)ethyl)-1H-indol-4-yl) 4,6-dimethylisophthalate

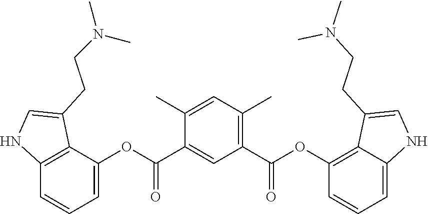 [SMILES: Cc1cc(C)c(C(=O)Oc2cccc3[nH]cc(CCN(C)C)c23)cc1C(=O)Oc1cccc2[nH]cc(CCN(C)C)c12]

The title compound, bis(3-(2-(dimethylamino)ethyl)-1H-indol-4-yl) 4,6-dimethylisophthalate, was prepared according to the protocol described in general procedure for Example 1 using DMF as solvent and isolated as a beige solid (0.045 g, 33% yield). ¹H NMR (400 MHz, CDCl₃) δ 9.25 (s, 1H), 8.54 (s, 2H), 7.31 (s, 1H), 7.22-7.08 (m, 4H), 6.89-6.82 (m, 4H), 2.88 (t, J=7.6 Hz, 4H), 2.76 (s, 6H), 2.58 (t, J=7.6 Hz, 4H), 2.07 (s, 12H). ESI-MS: measured m/z 505.3 [M+H]⁺. Purity by HPLC: 99.3% at 254 nm. ESI-MS: measured m/z 567.2 [M+H]⁺. Purity by HPLC: 98.8% at 254 nm.

### 1.G. bis(3-(2-(dimethylamino)ethyl)-1H-indol-4-yl) (1S,2S)-cyclopropane-1,2-dicarboxylate

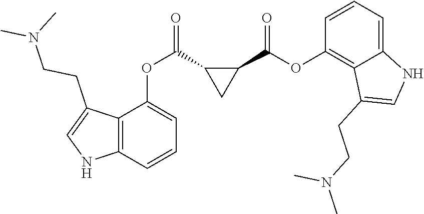 [SMILES: CN(C)CCc1c[nH]c2cccc(OC(=O)[C@H]3C[C@@H]3C(=O)Oc3cccc4[nH]cc(CCN(C)C)c34)c12]

The title compound, bis(3-(2-(dimethylamino)ethyl)-1H-indol-4-yl) (1S,2S)-cyclopropane-1,2-dicarboxylate, was prepared according to the protocol described in general procedure in Example 1 using DM F as solvent and isolated as a pale-yellow foam solid (0.030 g, 24% yield). The product was isolated as a white powder (0.065 g, 52% yield). ¹H NMR (400 MHz, CDCl₃) δ 8.11 (s, 2H), 7.24 (dd, J=8.2, 0.9 Hz, 2H), 7.18-7.11 (m, 2H), 7.01 (d, J=2.4 Hz, 2H), 6.86 (d, J=7.7 Hz, 2H), 3.02-2.91 (m, 4H), 2.79-2.71 (m, 2H), 2.68-2.58 (m, 4H), 2.32 (s, 12H), 1.87-1.78 (m, 2H). ESI-MS: measured m/z 503.2 [M+H]⁺. Purity by HPLC: 98.1% at 254 nm.

### Example 2: Alternative Preparation of Ester Psilocin Derivatives of Formula I

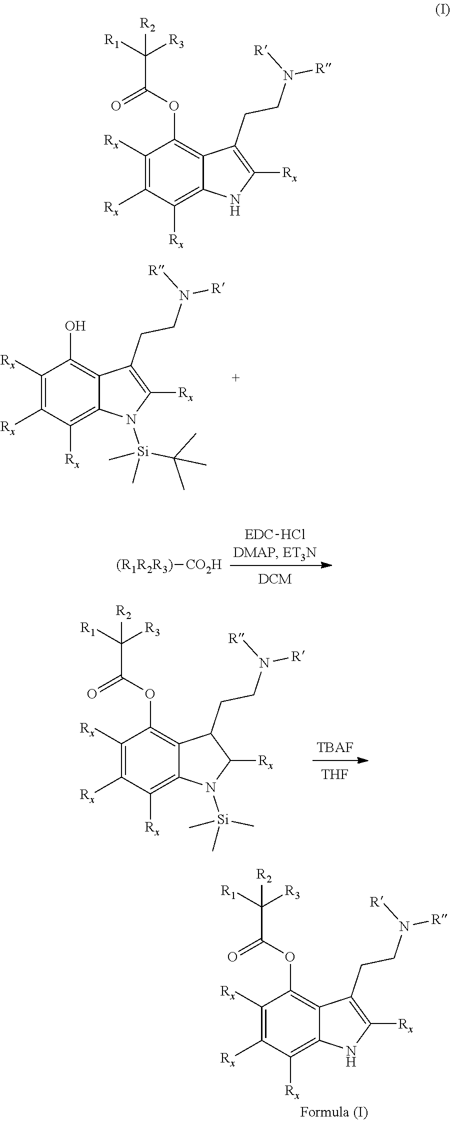

A series of ester psilocin derivatives are prepared according to the general reaction scheme above. Briefly, a solution comprising an amount of a protected psiolocin or psilocin derivative is prepared and reacted with an amount (e.g., a molar equivalent or slight excess) of a functionalized carboxylic acid along with 1-(3-DimethylaminopropyI)-3-ethylcarbodiimide hydrochloride (EDC-HCl), 4-dimethylaminopyridine (DMAP), and trimethylamine (Et₃N) in a suitable solvent (e.g., dichloromethane (DCM)) to generate the corresponding ester derivative. The ester derivative can be isolated prior to the deprotection step, or it can be converted in situ to the compound of Formula (I) by reaction with a deprotecting agent (e.g., tetrabutylammonium fluoride (TBAF)).

### General Procedure for Example 2

Under an inert atmosphere of nitrogen gas, N-protected psilocin or psilocin derivative (1 eq.) and functionalized carboxylic acid (1.6 eq.) were dissolved in anhydrous DCM (0.1 M). The resulting mixture was cooled and stirred at 0° C. before EDC HCl (1.6 eq.), DMAP (1.6 eq.), and Et₃ N (3 eq.) were added, respectively. The reaction was warmed to and maintained at a temperature of 25° C. for 24 hours. The reaction mixture was diluted with ethyl acetate (50 mL) upon completion. The organic phase was washed with brine (20 mL×3), it was dried over sodium sulfate, and the solvent was evaporated off. The crude reaction mixture was purified by normal phase column chromatography, running a mobile phase of 0% to 15% MeOH in DCM and the product containing fractions lyophilized to afford the desired product. Under an inert atmosphere of nitrogen gas, the appropriate TBS-protected acetate (1.0 eq.) was dissolved in THF (0.09-0.11 M). Tetrabutylammonium fluoride solution (2 eq., 1.0 M in THF) was added and the resulting mixture was stirred at room temperature (1-3 hours). Brine was added and extracted with CH₂Cl₂ or ethyl acetate. The organic layer dried over Na₂SO₄ and concentrated under reduced pressure. The crude reaction mixture was purified by normal phase column chromatography, running a mobile phase of 0% to 15% MeOH in DCM and the product containing fractions lyophilized to afford the desired product.

The following compounds illustrate embodiments in accordance with the above synthetic reaction strategy.

### 2.A. 3-(2-(dimethylamino)ethyl)-1H-indol-4-yl4-bromobenzoate

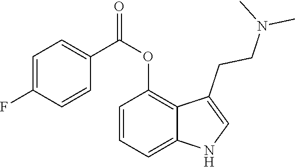 [SMILES: CN(C)CCc1c[nH]c2cccc(OC(=O)c3ccc(F)cc3)c12]

The title compound, 3-(2-(dimethylamino)ethyl)-1H-indol-4-yl4-fluorobenzoate, was prepared according to the general procedure for Example 2. The intermediate, 1-(tert-butyldimethylsilyl)-3-(2-(dimethylamino)ethyl)-1H-indol-4-yl 4-fluorobenzoate, isolated as white solid (0.150 g, 72% yield). ESI-MS: measured m/z 441.1 [M+H]⁺. The product obtained from general procedure D, isolated as an off-white solid (0.056 g, 50% yield). ¹H NMR (400 MHz, CD₃OD) δ 7.83-7.75 (m, 3H), 7.36-7.26 (m, 1H), 7.18-7.09 (m, 1H), 7.01 (s, 1H), 6.67 (d, J=8.0 Hz, 1H), 2.98 (t, J=7.1 Hz, 2H), 2.75 (t, J=7.5 Hz, 2H), 2.37-2.32 (m, 6H). ESI-MS: measured m/z 327.28 [M+H]⁺. Purity by HPLC: 99.9% at 254 nm

### 2.B. 3-(2-(dimethylamino)ethyl)-1H-indol-4-yl thiophene-2-carboxylate

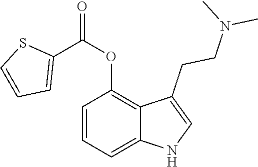 [SMILES: CN(C)CCc1c[nH]c2cccc(OC(=O)c3cccs3)c12]

The title compound, 3-(2-(dimethylamino)ethyl)-1H-indol-4-yl thiophene-2-carboxylate, was prepared according to the protocol described in general procedure for Example 2. The intermediate, 1-(tert-butyldimethylsilyl)-3-(2-(dimethylamino)ethyl)-1H-indol-4-yl thiophene-2-carboxylate, isolated as white solid (0.1119 g, 88% yield). ESI-MS: measured m/z 441.1 [M+H]⁺. The final product, shown above, was isolated as an off-white solid (0.016 g, 18% yield). ¹H NMR (400 MHz, CD₃OD) δ 8.14 (dd, J=3.8, 1.3 Hz, 1H), 7.97 (dd, J=5.0, 1.3 Hz, 1H), 7.37-7.32 (m, 2H), 7.25 (s, 1H), 7.21-7.14 (m, 1H), 6.86 (dd, J=7.7, 0.9 Hz, 1H), 3.28-3.20 (m, 2H), 3.11 (t, J=7.3 Hz, 2H), 2.62 (s, 6H). MS: measured m/z 315.30 [M+H]⁺. Purity by HPLC: 97.8% at 254 nm

### Example 3: Alternative Preparation of Ester Psilocin Derivatives of Formula II

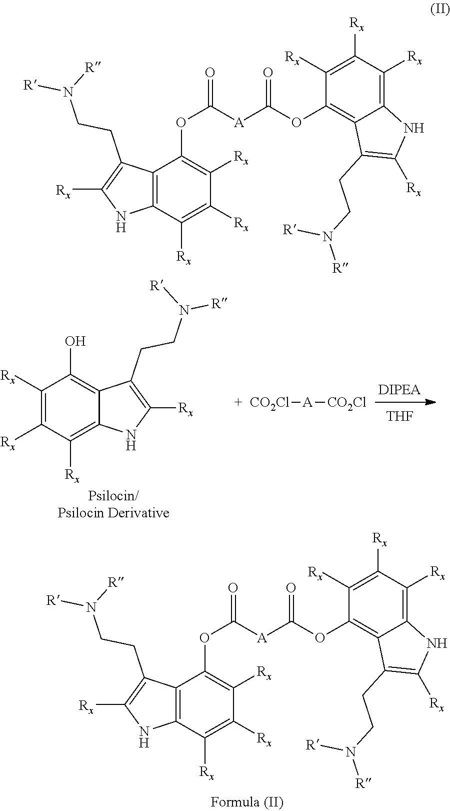

A series of ester psilocin derivatives are prepared according to the general reaction scheme above. Briefly, a solution comprising an amount of psilocin or a psilocin derivative (2 eq) is prepared and reacted with an amount of a functionalized acid chloride (1 eq) along with N,N-diisopropylethylamine (DIPEA) in a suitable solvent (e.g., tetrahydrofuran (THF)) to generate the corresponding ester derivative 2.

### General Procedure for Example 3

Under an inert atmosphere of argon gas, psilocin or a psilocin derivative (2 eq.) was dissolved in anhydrous THF (0.11-0.22 M) and cooled to 0° C. before neat DIPEA (4 eq.) was added. The resulting mixture was stirred at 0° C. for 5 minutes, followed by dropwise addition of functionalized acid chloride (1 eq.). The reaction was warmed to and maintained at a temperature of 25° C. for 2 hours. The reaction mixture was diluted with ethyl acetate (50 mL) upon completion. The organic phase was washed with brine (20 mL×3), dried over sodium sulfate, and the solvent was evaporated off. The crude reaction mixture was purified by Prep-HPLC, running a mobile phase of 90% to 80% H₂O (0.1% FA) in ACN (0.1% FA) over 60 minutes, and the product containing fractions lyophilized to afford the desired product.

### 3.A. bis(3-(2-(dimethylamino)ethyl)-1H-indol-4-yl) 2,2-dimethylmalonate

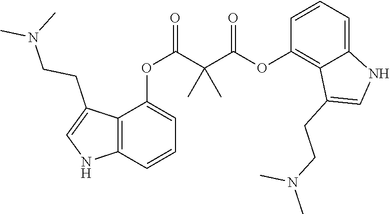 [SMILES: CN(C)CCc1c[nH]c2cccc(OC(=O)C(C)(C)C(=O)Oc3cccc4[nH]cc(CCN(C)C)c34)c12]

The title compound, bis(3-(2-(dimethylamino)ethyl)-1H-indol-4-yl) 2,2-dimethylmalonate, was prepared according to the protocol described in general procedure for Example 3 and isolated as a pale-yellow foam solid (0.025 g, 20% yield). ¹H NMR (400 MHz, CDCl₃) δ 8.16 (s, 2H), 7.20 (d, J=8.1 Hz, 2H), 7.11-7.04 (m, 2H), 7.00 (s, 2H), 6.83 (d, J=7.7 Hz, 2H), 2.96 (t, J=7.5 Hz, 4H), 2.62 (t, J=7.5 Hz, 4H), 2.23 (s, 12H), 1.90 (s, 6H). ESI-MS: measured m/z 505.3 [M+H]⁺. Purity by HPLC: 99.3% at 254 nm.

### 3.B. bis(3-(2-(dimethylamino)ethyl)-1H-indol-4-yl) glutarate

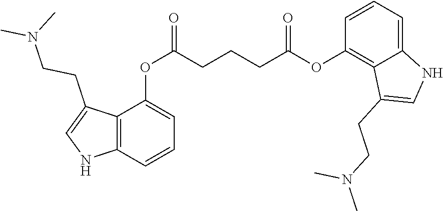 [SMILES: CN(C)CCc1c[nH]c2cccc(OC(=O)CCCC(=O)Oc3cccc4[nH]cc(CCN(C)C)c34)c12]

The title compound, bis(3-(2-(dimethylamino)ethyl)-1H-indol-4-yl) glutarate, was prepared according to the protocol described in general procedure for Example 3 and isolated as a pale-yellow foam solid (0.038 g, 34% yield). ¹H NMR (400 MHz, CDCl₃) δ 8.11 (s, 2H), 7.21 (dd, J=8.2, 0.9 Hz, 2H), 7.16-7.09 (m, 2H), 6.98 (d, J=2.3 Hz, 2H), 6.82 (dd, J=7.7, 0.9 Hz, 2H), 2.95-2.85 (m, 8H), 2.63-2.55 (m, 4H), 2.33-2.25 (m, 14H). ESI-MS: measured m/z 505.1 [M+H]⁺. Purity by HPLC: 99.4% at 254 nm.

### 3.C. bis(3-(2-(dimethylamino)ethyl)-1H-indol-4-yl) adipate

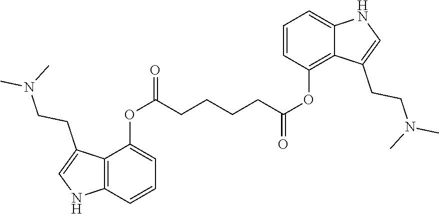

The title compound, bis(3-(2-(dimethylamino)ethyl)-1H-indol-4-yl) adipate, was prepared according to the protocol described in general procedure for Example 3 and isolated as a pale-yellow foam solid (0.030 g, 24% yield). ¹H NMR (400 MHz, CDCl₃) δ 8.11 (s, 2H), 7.20 (dd, J=8.1, 0.9 Hz, 2H), 7.15-7.09 (m, 2H), 6.97 (d, J=2.3 Hz, 2H), 6.80 (dd, J=7.6, 0.9 Hz, 2H), 2.95-2.87 (m, 4H), 2.81-2.72 (m, 4H), 2.64-2.54 (m, 4H), 2.30 (s, 12H), 2.01-1.92 (m, 4H). ESI-MS: measured m/z 519.3 [M+H]⁺. Purity by HPLC: 99.5% at 254 nm

### Example 4: Alternative Preparation of Ester Psilocin Derivatives of Formula II

### Generalized Reaction Scheme

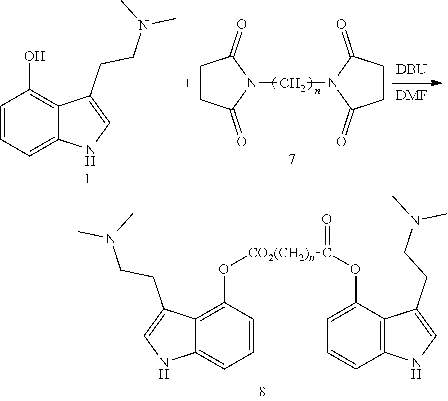

### Illustrative Reaction Scheme to Generate Compound (8), bis(3-(2-(dimethylamino)ethyl)-1H-indol-4-yl) succinate)

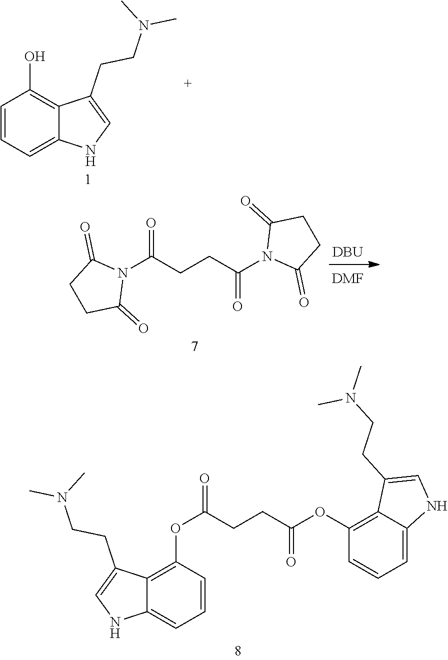

Adapting the general reaction scheme utilizing various succinimides above, the illustrative scheme to prepare title compound (8), bis(3-(2-(dimethylamino)ethyl)-1H-indol-4-yl) succinate, was prepared according to the following protocol; under an inert atmosphere of argon gas, psilocin (2 eq.) and a functionalized succinate (1 eq.) were dissolved in anhydrous DMF (0.25 M). The resulting mixture was stirred at room temperature before neat DBU (2 eq.) was added. The reaction was heated to and maintained at a temperature of 60° C. for 3 hours and subsequently cooled to room temperature. Once at ambient temperature, the reaction mixture was diluted with ethyl acetate (50 mL). The organic phase was washed with brine (20 mL×3), it was dried over sodium sulfate, and the solvent was evaporated off. The crude reaction mixture was purified by normal phase column chromatography, running a mobile phase of 0% to 15% (2 M NH3 in MeOH) in DCM and the product containing fractions lyophilized to afford the desired product. The product was isolated as a pale-yellow powder (0.015 g, 12% yield). ¹H NMR (400 MHz, DMSO-d6) δ 10.91 (s, 2H), 7.76 (d, J=8.2 Hz, 2H), 7.63 (s, 2H), 7.09 — 7.02 (m, 2H), 6.61 (d, J=7.8 Hz, 2H), 3.38 (s, 4H), 2.94 (t, J=6.9 Hz, 4H), 2.65 (t, J=7.1 Hz, 5H), 2.26 (s, 12H). ESI-MS: measured m/z 491.1 [M+H]⁺ . Purity by HPLC: 98.3% at 254 nm.

### Example 5: Compound Stability

Compound stability was evaluated as follows.

IP-One incubation. CHO-K1 cells expressing recombinant Serotonin receptor 5-HT_{2A} (Perkin Elmer, Waltham, MA), were seeded in 96-well plates (˜80,000 cells/well) and treated with 28 μL (per well) of stimulation medium (IP-One HTRF® kit) containing test compounds (2 μM) for 1 hr in a CO₂ incubator (5%) at 37° C. The cell-treated medium from 3 wells were pooled together to provide 40 μL for LC-MS analysis. Aliquots for each compound were collected at time zero (T₀, 100% intact test compound) and 60-min. The incubated medium (40 μL) was quenched with 120 μL of acetonitrile containing 0.5 μM glyburide (IS), and the supernatant was collected by centrifugation (2500 rpm, 15 min.). LC-MS was performed using an Agilent MSD (electrospray in positive ionization) equipped with a Kinetic C₁₈ reverse-column. The percent of test compound remaining is calculated using the ratio of the peak areas of intact test compound at 60 min. over T₀. The percent of psilocin release is calculated by comparing the peak area of psilocin at 60 min. (for test compound) vs. peak area at T₀ (2 μM psilocin). Results are summarized in Table 1, (rel to % parent detected; and % psilocin detected at 1 hr).

TABLE 1
Stability of compounds

| | % | % |
|---|---|---|
| | parent | psilocin |
| Compound | compound | release |
| 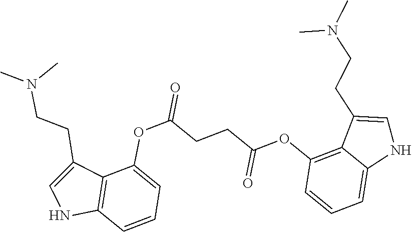 [SMILES: CN(C)CCc1c[nH]c2cccc(OC(=O)CCC(=O)Oc3cccc4[nH]cc(CCN(C)C)c34)c12] | 61.7 | 5.8 |
| 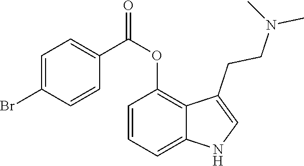 [SMILES: CN(C)CCc1c[nH]c2cccc(OC(=O)c3ccc(Br)cc3)c12] | 9.1 | 49.0 |
| 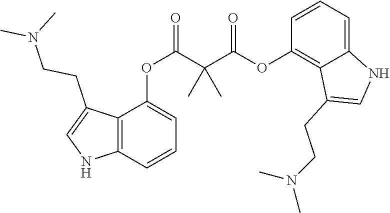 [SMILES: CN(C)CCc1c[nH]c2cccc(OC(=O)C(C)(C)C(=O)Oc3cccc4[nH]cc(CCN(C)C)c34)c12] | 86.8 | 6.5 |
| 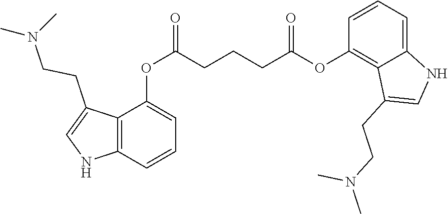 [SMILES: CN(C)CCc1c[nH]c2cccc(OC(=O)CCCC(=O)Oc3cccc4[nH]cc(CCN(C)C)c34)c12] | 71.5 | 11.8 |
| 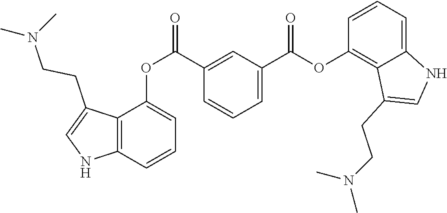 [SMILES: CN(C)CCc1c[nH]c2cccc(OC(=O)c3cccc(C(=O)Oc4cccc5[nH]cc(CCN(C)C)c45)c3)c12] | 52.1 | 16.5 |
| 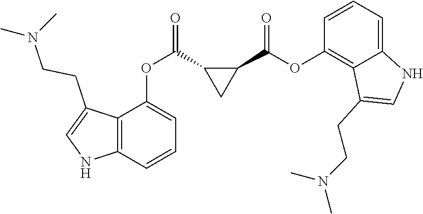 [SMILES: CN(C)CCc1c[nH]c2cccc(OC(=O)[C@H]3C[C@@H]3C(=O)Oc3cccc4[nH]cc(CCN(C)C)c34)c12] | 76.1 | 59.6 |
| 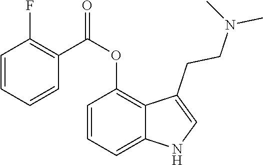 [SMILES: CN(C)CCc1c[nH]c2cccc(OC(=O)c3ccccc3F)c12] | 11.4 | 102.6 |
| 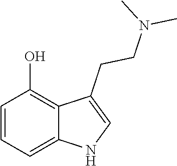 | 87.4 | 87.4 |
| (Psilocin) | | |

Fasted state simulated gastric fluid (FaSSGF). FaSSGF was prepared according to the manufacturer instructions (Biorelevant, UK). Test compounds (2 μL of 200 μM solution) were added to a 96-well plate containing 198 μL FaSSGF per well. The mixture was incubation in a thermomixer at 37° C. for 60 min. Sample aliquots were removed (40 μL, from T₀ and 60 min.) and quenched with acetonitrile containing 0.5 μM glyburide (IS), and the supernatant collected by centrifugation (2500 rpm, 15 min.). LC-MS was performed using an Agilent MSD (electrospray in positive ionization) equipped with a Kinetic C₁₈ reverse-column. The percent of test compound remaining was calculated using the ratio of the peak area of intact test compound at 60 min. over T₀. The percent of psilocin release was calculated by comparing the peak area of psilocin at 60 min, (for test compound) vs. peak area at T₀ (2 μM psilocin). Table 2 summarizes the results.

TABLE 2
Stability of compounds

| | % parent | % |
|---|---|---|
| Compound | compound | psilocin |
| 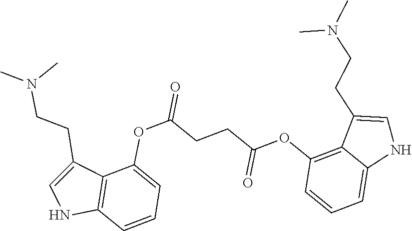 [SMILES: CN(C)CCc1c[nH]c2cccc(OC(=O)CCC(=O)Oc3cccc4[nH]cc(CCN(C)C)c34)c12] | 97.1 | 0.0 |
| 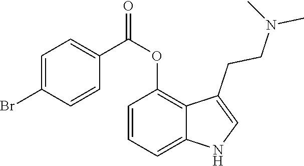 [SMILES: CN(C)CCc1c[nH]c2cccc(OC(=O)c3ccc(Br)cc3)c12] | 93.8 | 0.0 |
| 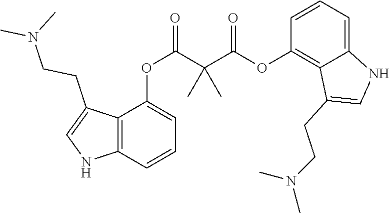 [SMILES: CN(C)CCc1c[nH]c2cccc(OC(=O)C(C)(C)C(=O)Oc3cccc4[nH]cc(CCN(C)C)c34)c12] | 94.7 | 0.0 |
| 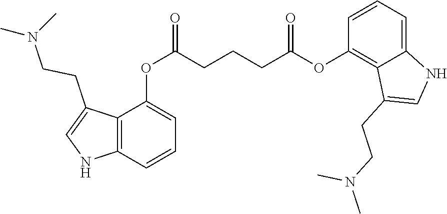 [SMILES: CN(C)CCc1c[nH]c2cccc(OC(=O)CCCC(=O)Oc3cccc4[nH]cc(CCN(C)C)c34)c12] | 94.5 | 0.0 |
| 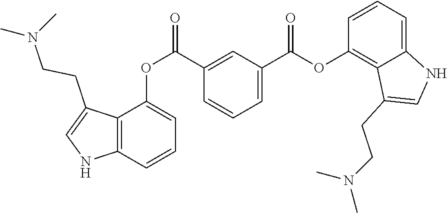 [SMILES: CN(C)CCc1c[nH]c2cccc(OC(=O)c3cccc(C(=O)Oc4cccc5[nH]cc(CCN(C)C)c45)c3)c12] | 93.5 | 0.0 |
| 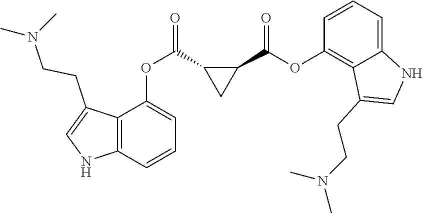 [SMILES: CN(C)CCc1c[nH]c2cccc(OC(=O)[C@H]3C[C@@H]3C(=O)Oc3cccc4[nH]cc(CCN(C)C)c34)c12] | 95.5 | 0.0 |
| 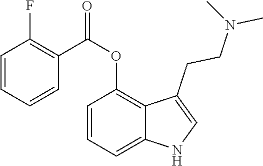 [SMILES: CN(C)CCc1c[nH]c2cccc(OC(=O)c3ccccc3F)c12] | 97.5 | 0.0 |
| 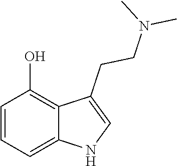 | 72.2 | — |
| (Psilocin) | | |

### Example 6: 5HT2A Receptor Displacement

Test compounds were evaluated for binding activity against human serotonin 5HT2A receptor using a radioligand displacement assay. Briefly, in a series of wells in a 96-well microplate, 15 μg of 5HT2A membrane (prepared from a commercial HEK293 cell line (PerkinElmer)) were pre-incubated with increasing concentrations of test compound (0-10,000 nM) for 30 min. at 30° C. After incubation, either 5 nM [³H]-ketanserin (PerkinElmer, NET12333250UC) or 3 nM [³H]-LSD (American Radiolabeled Chemicals Inc, ART0896) was added to the reaction well and was incubated for 60 min. at 30° C. For each assay performed, at least two wells were dedicated for a standard and a non-specific binding control, where the latter was determined in the presence of cold/unlabeled Ketanserin (Cerilliant, L-001) or LSD (Cerilliant, L-001, TK #61-1779) at a final concentration of 10 μM per reaction. The total reaction volume was 400 μL/well in reaction buffer (50 mM Tris-Cl, pH 7.4, 4 mM CaCl₂, and 0.1% ascorbic acid), and was stopped by transferring the mixture to a Multiscreen 96-well filter plate (Millipore Sigma, MSFCNXB50) pretreated with 0.25% of PEI in 50 mM Tris-Cl, pH 7.4. The mixture was then filtered and washed ten times with 200 μL of chilled wash buffer (50 mM Tris-Cl, pH 7.4) using a vacuum manifold (Millipore MultiScreenHTS, MSVNHTS00). Post-wash, the plates were dried in an incubator (at 50° C.) and are treated with 40 μL of scintillation cocktail (Ultima GoldTM XR) to detect and quantify signal using a 1450 MicroBeta scintillation counter (PerkinElmer). Results are summarized in Table 3, with activity reported relative to psilocin.

TABLE 3
Displacement activity

| | Activity relative to psilocin |
|---|---|
| | + = lower, |
| | ++ = same, |
| Compound | +++ = higher |
| 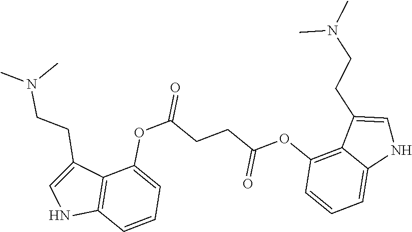 [SMILES: CN(C)CCc1c[nH]c2cccc(OC(=O)CCC(=O)Oc3cccc4[nH]cc(CCN(C)C)c34)c12] | +++ |
| 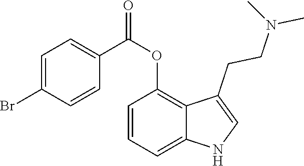 [SMILES: CN(C)CCc1c[nH]c2cccc(OC(=O)c3ccc(Br)cc3)c12] | ++ |
| 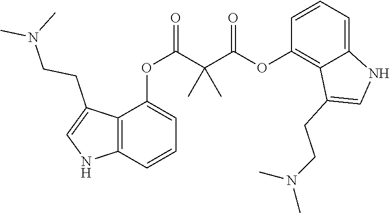 [SMILES: CN(C)CCc1c[nH]c2cccc(OC(=O)C(C)(C)C(=O)Oc3cccc4[nH]cc(CCN(C)C)c34)c12] | +++ |
| 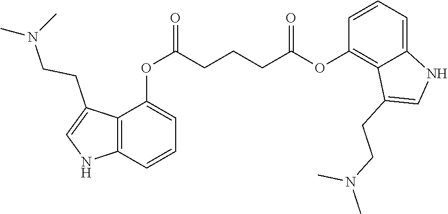 [SMILES: CN(C)CCc1c[nH]c2cccc(OC(=O)CCCC(=O)Oc3cccc4[nH]cc(CCN(C)C)c34)c12] | +++ |
| 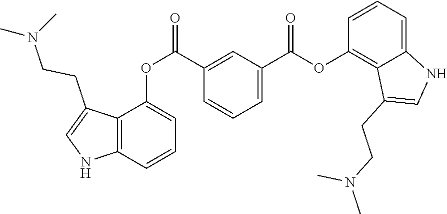 [SMILES: CN(C)CCc1c[nH]c2cccc(OC(=O)c3cccc(C(=O)Oc4cccc5[nH]cc(CCN(C)C)c45)c3)c12] | +++ |
| 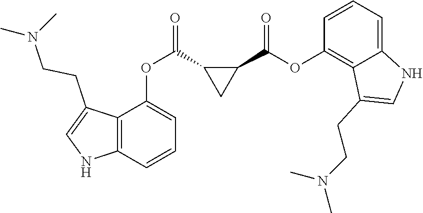 [SMILES: CN(C)CCc1c[nH]c2cccc(OC(=O)[C@H]3C[C@@H]3C(=O)Oc3cccc4[nH]cc(CCN(C)C)c34)c12] | +++ |
| 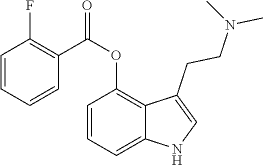 [SMILES: CN(C)CCc1c[nH]c2cccc(OC(=O)c3ccccc3F)c12] | ++ |
| 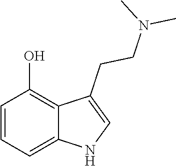 | ++ |
| (Psilocin) | |

### Example 7: p-Arrestin Recruitment

The test compounds can be evaluated for β-arrestin recruitment activity utilizing commercially available assay systems. The PathHunter® GPCR (DiscoverX) uses proprietary technology and cells that are engineered to co-express the ProLink (PK) tagged GPCR and the Enzyme Acceptor (EA) tagged β-arrestin. Activation of the GPCR-PK induces β-arrestin-EA recruitment, facilitating complementation of the two β-galactosidase enzyme fragments (EA and PK). The resulting functional enzyme hydrolyzes a substrate to generate a chemiluminescent signal.

Cells (e.g., U2OS β-arrestin-EA cells) expressing Serotonin receptor 5-HT_{2A/2C}-PK are grown according to the manufacturer's instructions (AssayComplete™ Cell Culture Kit 112 (DiscoverX)), with hygromycin and geneticin (G418). Prior to the assay, 40,000 cells/well are seeded in cell culture-treated, flat bottom 96-well plates (Greiner 82050-736) in plating media (AssayComplete™ Cell Plating 19 DiscoverX). The cells are washed once with PBS and incubated with 50 μL PBS containing compounds of interest then incubated at 37° C., 5% CO₂ for 90 min. Following incubation, 25 μL of the detection solution (PathHunter® Detection Kit, DiscoverX) is added to each well and plates are incubated for 1 hour at room temperature β-arrestin recruitment is quantified by measuring the generated luminescence signal using a microplate reader (Spark, Tecan). Test compounds can show good levels of β-arrestin recruitment activity, relative to controls and reference compounds.

### Example 8: Pharmacokinetics

The single-dose pharmacokinetics of test compounds were analyzed relative to control (psilocin) in C57BL6 mice via oral (PO) administration. Test compound was dissolved in DMSO and transferred to a 15-mL tube, followed by addition of Tween-80, PEG-400, and water to final volume. The resulting formulation (0.3 mg/mL compound, in DMSO (1%), Tween-80 (5%), and PEG-400 (25%) in water (69%)) was vortexed for 2 min. and mixed by inversion.

Male C57BL6 mice (3× per compound, (at a weight ˜20g)) received a single dose (oral) of test compound at 3.0 mg/kg. Blood samples were collected pre-dose and at 0.083, 0.25, 0.5, 1, 2, 4, 8, 24-hour post-dose to yield approximately 20 μL of pooled plasma (3× mice) per time point. Brain tissues were collected at 2- and 8-hr post-dose. Brain was flushed with saline solution using a perfusion needle through the ascending aorta of the ventricle to remove any remaining blood prior to collection. Plasma and brain homogenate samples were stored at −80° C. prior to extraction for LC-MS/MS analysis.

Samples were analyzed by LC-MS/MS to quantify the amounts of test compound and control (psilocin). The calculation of the pharmacokinetic (PK) parameters (AUCₜ, AUC_{inf}, Cₘₐₓ, Tₘₐₓ, T_{1/2}, and Kₑₗ) can be performed using a non-compartmental analysis (trapezoidal method).

Control (psilocin). The method was able to quantify psilocin in brain homogenate, and an estimate for psilocin plasma concentration (of 5 ng/mL, based on other pharmacokinetic data) was used to determine a brain/plasma (B/P) ratio as an indicator of brain penetration. Psilocin concentration in the brain ranged from 40 to 78 ng/g at 2-h post-dose, resulting in a mean B/P ratio of 11.2±4.3. No psilocin in the brain was detected at 8-h post-dose. The B/P data and ratio can be used to compare brain penetration by the compounds of the disclosure.

### Test Compounds

### Test Compound A. bis(3-(2-(dimethylamino)ethyl)-1H-indol-4-yl) 2,2-dimethylmalonate

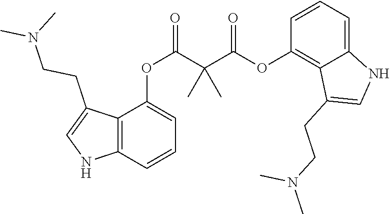 [SMILES: CN(C)CCc1c[nH]c2cccc(OC(=O)C(C)(C)C(=O)Oc3cccc4[nH]cc(CCN(C)C)c34)c12]

Analysis of any psilocin in plasma following administration of this compound did not provide reliable data; however, the LC-MSMS method detected the unchanged parent compound in all plasma samples (from 1.4 to 4.2 ng/mL), allowing for estimates of PK parameters for intact compound. The PK profile and the B/P ratio suggested a quick absorption of the compound, but with a long elimination phase. This translated into a delayed brain penetration of the parent compound. The absence of the release of psilocin also correlated with the in vitro assays which detected little to no psilocin, indicating the potential for this test compound to be developed as a novel therapeutic compound.

### Test Compound B. bis(3-(2-(dimethylamino)ethyl)-1H-indol-4-yl) glutarate

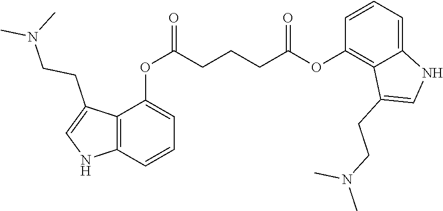 [SMILES: CN(C)CCc1c[nH]c2cccc(OC(=O)CCCC(=O)Oc3cccc4[nH]cc(CCN(C)C)c34)c12]

Analysis and detection of psilocin and test compound in plasma and brain was performed, with the results indicating that the test compound possesses a good PK profile as a psilocin prodrug. For example, following administration of the test compound, no parent compound was detected in any of the plasma or brain samples; however psilocin was detected in quantifiable amounts. The PK analysis showed a profile that was comparable to that of psilocybin dosed in humans (at 0.3 mg/kg), with similar PK parameters. Following administration of the test compound, psilocin was effectively penetrating to the brain with a calculated B/P ratio 6 times greater than that for the B/P ratio associated with the administration of psilocin. Moreover, psilocin was detectable in brain for up to 8 hr. following administration of the test compound.

TABLE 4
PK Summary

| | Parent PK | | | Psilocin Released | | | | | | |
|---|---|---|---|---|---|---|---|---|---|---|
| | | | [brain] | | | | [plasma] | [brain] | [plasma] | [brain] |
| | Cmax | AUC | 2 h | Cmax | AUC | T½ | 2 h | 2 h | 8 h | 8 h |
| CMP | ng/mL | ng/mL*h | ng/mL | ng/mL | ng/mL*h | h | ng/mL | ng/mL | ng/mL | ng/mL |
| Psil. | 5.0* | — | 54 | — | — | — | — | 54 | — | 0 |
| A | 4.2 | 39 | 0.5** | 0 | 0 | — | 0 | 0 | 0 | 0 |
| B | 0 | 0 | 0 | 5.2 | 43 | 3.0 | 4.2 | 292 | 1.9 | 21.1 |

*(estimated)
**[brain] at 8 h is 6 ng/mL.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of Formula (I): 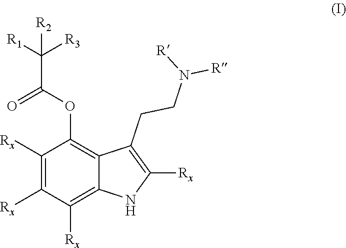
or a pharmaceutically acceptable salt thereof, wherein
R₁ is absent, or hydrogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which is optionally substituted with one or more of halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
R₂ and R₃ are independently hydrogen, deuterium, halogen, C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which is optionally substituted with one or more of halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl; or
R₂ and R₃ together with the carbon atom to which they are attached form a C₃-C₂₀ cycloalkyl, aryl, or heteroaryl, any of which is optionally substituted with one or more halogen, C₁-C₆ alkyl, C₂-C₆ alkenyl, C₁-C₆ haloalkyl, C₃-C₁₂ cycloalkyl, C₃-C₁₂ heterocyclyl, aryl, or heteroaryl;
R′ and R″ are independently hydrogen, C₁-C₆ alkyl, deuterated C₁-C₆ alkyl, C₂-C₆ alkenyl, C₂-C₆ alkynyl, or R′ and R″ together with the nitrogen atom to which they are attached form a C₃-C₁₂ heterocyclyl or heteroaryl; and
each Rx is independently hydrogen, —OH, methyl, methoxy, C₁-C₃ alkyl, C₁-C₆ haloalkyl, deuterated C₁-C₃ alkyl, C₁-C₃ alkoxy, —C(═O)OH, —C(═O)NR₂R₃, or halogen;
wherein the compound is not

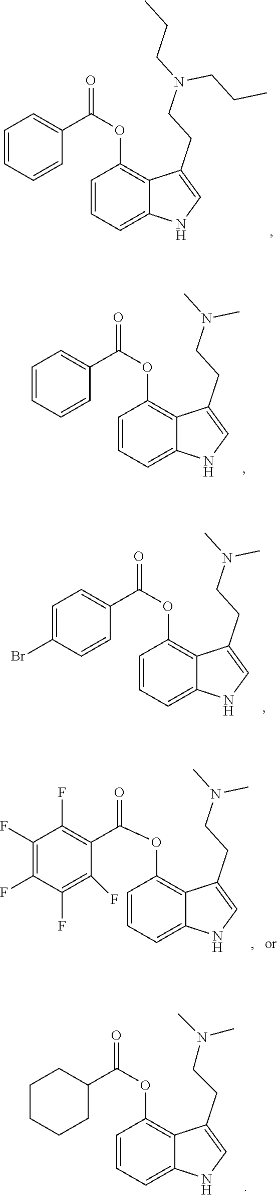 [SMILES: CCCN(CCC)CCc1c[nH]c2cccc(OC(=O)c3ccccc3)c12 ; CN(C)CCc1c[nH]c2cccc(OC(=O)c3ccccc3)c12 ; CN(C)CCc1c[nH]c2cccc(OC(=O)c3ccc(Br)cc3)c12 ; CN(C)CCc1c[nH]c2cccc(OC(=O)c3c(F)c(F)c(F)c(F)c3F)c12 ; CN(C)CCc1c[nH]c2cccc(OC(=O)C3CCCCC3)c12]

2. The compound of claim 1, wherein R′ and R″ are independently a C₁-C₆ alkyl.

3. The compound of claim 1, wherein each Rₓ is independently hydrogen, —OH, methyl, methoxy, or halogen.

4. The compound of claim 1, wherein R₁ is absent and R₂ and R₃ together with the carbon atom to which they are attached form a C₃-C₂₀ cycloalkyl, C₃-C₂₀ heterocyclyl, aryl, or heteroaryl, any of which is optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl.

5. The compound of claim 1, wherein
(i) one of R₂ or R₃ is hydrogen, and the other of R₂ or R₃ is C₂-C₆ alkenyl, C₂-C₆ alkynyl, C₁-C₆ alkoxy, or C₃-C₁₂ cycloalkyl; or
(ii) R₂ and R₃ together with the carbon atom to which they are attached form a C₃-C₁₂ cycloalkyl that is optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl.

6. The compound of claim 1, wherein the compound comprises at least one deuterium atom.

7. The compound of claim 1, comprising having a structure according to Formula (Ib): 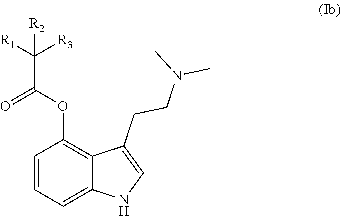
or a pharmaceutically acceptable salt thereof, wherein
R₁ is absent and R₂ and R₃ together with the carbon atom to which they are attached form a C₃-C₂₀ cycloalkyl, aryl, or heteroaryl, any of which is optionally substituted with one or more of halogen, C₁-C₆ alkyl, C₂-C₆ alkenyl, C₁-C₆ haloalkyl, C₃-C₁₂ cycloalkyl, C₃-C₁₂ heterocyclyl, aryl, or heteroaryl.

8. The compound of claim 1, selected from:
3-(2-(dimethylamino)ethyl)-1H-indol-4-yl furan-3-carboxylate;
3-(2-(dimethylamino)ethyl)-1H-indol-4-yl 2-fluorobenzoate;
3-(2-(dimethylamino)ethyl)-1H-indol-4-yl 3 -fluorobenzoate;
3-(2-(dimethylamino)ethyl)-1H-indol-4-yl 4-fluorobenzoate; or 3-(2-(dimethylamino)ethyl)-1H-indol-4-yl thiophene-2-carboxylate,
or a pharmaceutically acceptable salt thereof.

9. A pharmaceutical composition, comprising the compound of claim 1 and a pharmaceutically acceptable carrier.

10. A method for treating one or more conditions that are responsive to serotonin receptor activation, comprising administering to a subject in need thereof an effective amount of the compound of claim 1.

11. A method for treating a neurological disease, comprising administering to a subject in need thereof an effective amount of the compound of claim 1.

12. The method of claim 11, wherein the neurological disease is a neurodegenerative disease, stupor and coma, dementia, seizure, sleep disorder, trauma, infection, neoplasm, neuro-ophthalmological condition, movement disorder, demyelinating disease, spinal cord disorder, disorder of peripheral nerves, muscle and neuromuscular junctions, psychiatric disorder, pain, or a disease associated with pain.

13. The method according to claim 12, wherein the psychiatric disorder is: an anxiety disorder including acute stress disorder agoraphobia, generalized anxiety disorder, obsessive-compulsive disorder, panic disorder, posttraumatic stress disorder, separation anxiety disorder, social phobia, or specific phobia; a childhood disorder including attention-deficit/hyperactivity disorder, conduct disorder, or oppositional defiant disorder; an eating disorder including anorexia nervosa or bulimia nervosa; a mood disorder including depression, bipolar disorder, cyclothymic disorder, dysthymic disorder, or major depressive disorder; a personality disorder including antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, or schizotypal personality disorder; a psychotic disorder including brief psychotic disorder, delusional disorder, schizoaffective disorder, schizophreniform disorder, schizophrenia, or shared psychotic disorder; a substance-related disorder including alcohol dependence, amphetamine dependence, cannabis dependence, cocaine dependence, hallucinogen dependence, inhalant dependence, nicotine dependence, opioid dependence, phencyclidine dependence, or sedative dependence; an adjustment disorder, autism, delirium, dementia, multi-infarct dementia, a learning or memory disorder including amnesia or age-related memory loss; or Tourette's disorder.

14. The method of claim 12, wherein the neurological disease is pain, or a disease associated with pain.

15. A compound of Formula (II): 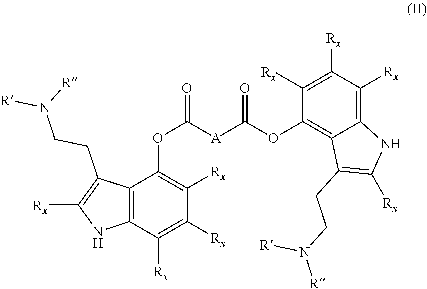
or a pharmaceutically acceptable salt thereof, wherein
A is C₁-C₁₂ alkyl, C₂-C₁₂ alkenyl, C₂-C₁₂ alkynyl, C₁-C₁₂ alkoxy, C₁-C₁₂ haloalkyl, C₃-C₂₀ cycloalkyl, C₃-C₂₀ heterocyclyl, aryl, or heteroaryl, any of which is optionally substituted with one or more halogen, C₁-C₆ alkyl, C₂-C₆ alkenyl, C₁-C₆ alkoxy, C₁-C₆ haloalkyl, C₃-C₁₂ cycloalkyl, C₃-C₁₂ heterocyclyl, aryl, or heteroaryl;
each R′ and R″ is independently hydrogen, C₁-C₆ alkyl, deuterated C₁-C₆ alkyl, C₂-C₆ alkenyl, C₂-C₆ alkynyl, or R′ and R″ together with the nitrogen atom to which they are attached form a C₃-C₁₂ heterocyclyl or heteroaryl; and
each Rₓ is independently hydrogen, —OH, methyl, methoxy, C₁-C₃ alkyl, C₁-C₆ haloalkyl, deuterated C₁-C₃ alkyl, C₁-C₃ alkoxy, —C(═O)OH, -C(═O)NR₂R₃, or halogen.

16. The compound of claim 15, wherein R′ and R″ are independently a C₁-C₆ alkyl.

17. The compound of claim 15, wherein each Rₓ is independently hydrogen, —OH, methyl, methoxy, or halogen.

18. The compound of claim 15any of claims 15, wherein A is C₁-C₆ alkyl, C₂-C₆ alkenyl, C₂-C₆ alkynyl, C₁-C₆ alkoxy, or C₃-C₁₂ cycloalkyl.

19. The compound of claim 15, wherein the compound comprises at least one deuterium atom.

20. The compound of claim 15, comprising having a structure according to Formula (IIb): 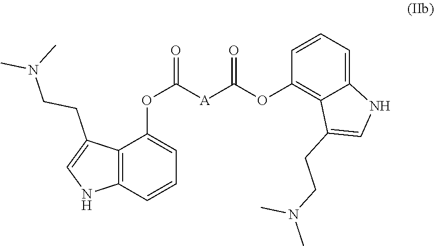
or a pharmaceutically acceptable salt thereof, wherein
A is C₁-C₆ alkyl, C₂-C₆ alkenyl, C₂-C₆ alkynyl, C₁-C₆ alkoxy, C₃-C₁₂ cycloalkyl, C₃-C₁₂ heterocyclyl, aryl, or heteroaryl.

21. The compound of claim 15, selected from:
bis(3-(2-(dimethylamino)ethyl)-1H-indol-4-yl) isophthalate;
bis(3-(2-(dimethylamino)ethyl)-1H-indol-4-yl) 4,6-dimethylisophthalate;
bis(3-(2-(dimethylamino)ethyl)-1H-indol-4-yl) (1S,2S)-cyclopropane-1,2-dicarboxylate;
bis(3-(2-(dimethylamino)ethyl)-1H-indol-4-yl) 2,2-dimethylmalonate;
bis(3-(2-(dimethylamino)ethyl)-1H-indol-4-yl) succinate;
bis(3-(2-(dimethylamino)ethyl)-1H-indol-4-yl) glutarate; or
bis(3-(2-(dimethylamino)ethyl)-1H-indol-4-yl) adipate,
or a pharmaceutically acceptable salt thereof.

22. A pharmaceutical composition, comprising the compound of claim 15 and a pharmaceutically acceptable carrier.

23. A method for treating one or more conditions that are responsive to serotonin receptor activation, comprising administering to a subject in need thereof an effective amount of the compound of claim 15.

24. A method for treating a neurological disease, comprising administering to a subject in need thereof an effective amount of the compound of claim 15.

25. The method of claim 24, wherein the neurological disease is a neurodegenerative disease, stupor and coma, dementia, seizure, sleep disorder, trauma, infection, neoplasm, neuro-ophthalmological condition, movement disorder, demyelinating disease, spinal cord disorder, disorder of peripheral nerves, muscle and neuromuscular junctions, psychiatric disorder, pain, or a disease associated with pain.

26. The method of claim 25, wherein the psychiatric disorder is: an anxiety disorder including acute stress disorder agoraphobia, generalized anxiety disorder, obsessive-compulsive disorder, panic disorder, posttraumatic stress disorder, separation anxiety disorder, social phobia, or specific phobia; a childhood disorder including attention-deficit/hyperactivity disorder, conduct disorder, or oppositional defiant disorder; an eating disorder including anorexia nervosa or bulimia nervosa; a mood disorder including depression, bipolar disorder, cyclothymic disorder, dysthymic disorder, or major depressive disorder; a personality disorder including antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, or schizotypal personality disorder; a psychotic disorder including brief psychotic disorder, delusional disorder, schizoaffective disorder, schizophreniform disorder, schizophrenia, or shared psychotic disorder;
a substance-related disorder including alcohol dependence, amphetamine dependence, cannabis dependence, cocaine dependence, hallucinogen dependence, inhalant dependence, nicotine dependence, opioid dependence, phencyclidine dependence, or sedative dependence; an adjustment disorder, autism, delirium, dementia, multi-infarct dementia, a learning or memory disorder including amnesia or age-related memory loss; or Tourette's disorder.

27. The method of claim 25, wherein the neurological disease is pain, or a disease associated with pain.
